(19) United States
(12) Patent Application Publication
(10) Pub. No.: US 2026/0315814 A1
(43) Pub. Date: Oct. 15, 2026

(54) IMPLANT DRIVERS AND METHODS OF USE THEREWITH

(71) Applicant: Edwards Lifesciences Corporation, Irvine, CA (US)

(72) Inventors: Ido Avrahamov, Tel-Aviv (IL); Ofry Efraim Yossef, Tel-Aviv (IL); Carmel Peleg, Pardes Hana-Karkur (IL); Ido Halabi, Givatayim (IL); Haim Brauon, Beit Dagan (IL)

(21) Appl. No.: 19/693,216

(22) Filed: May 29, 2026

Related U.S. Application Data
(63) Continuation of application No. PCT/US2024/055332, filed on Nov. 11, 2024.
(60) Provisional application No. 63/604,878, filed on Nov. 30, 2023.

(51) Int. Cl. A61N 1/375; A61B 17/00; A61B 17/34

(52) U.S. Cl. CPC A61N 1/37518; A61B 17/3468; A61N 1/37512; A61B 2017/00292

(57) ABSTRACT

An apparatus for use with a heart of a subject comprises a driver, comprising a shaft and a drive head disposed at a distal end of the shaft. The drive head comprises fingers and a slot between the fingers. The driver is configured to engage an anchor head of a tissue anchor such that, during insertion of the driver into the anchor head, the fingers deflect radially to inhibit axial movement of the drive head relative to the anchor, and the slot abuts a protrusion of the anchor head in a manner that delivers torque to the anchor head upon rotation of the shaft. Other embodiments are also described.

### CROSS-REFERENCE TO RELATED APPLICATION

The present application is a Continuation of International Patent Application No. PCT/US2024/055332 to Avrahamov et al., filed Nov. 11, 2024, and titled “Implant drivers and methods of use therewith,” which published as WO2025/117165, and which claims priority to U.S. Patent Application No. 63/604,878, to Avrahamov et al., filed Nov. 30, 2023, and titled “Anchor drivers and methods of use therewith”, the entire disclosure of which is incorporated by reference for all purposes.

### TECHNICAL FIELD

Some implementations of the present invention relate in general to implantation techniques. More specifically, some implementations of the present invention relate to techniques for transferring torque to implants.

### BACKGROUND

In recent years, the number of transcatheter cardiac surgeries and medical processes in mammalian, and specifically human, hearts, has been increasing. Many such procedures require insertion of an implant and/or a tissue anchor into the cardiac tissue by a driver (e.g., an anchor driver) which typically extends transluminally within a catheter.

In many prior art drivers, the drive head is secured, by welding, to a longitudinal shaft extending out of the body. Welding may be unreliable in certain circumstances, and may be less predictable in its behavior under strained conditions.

There is thus a need for improved techniques and devices for driving tissue anchors into the tissue of the heart, or otherwise applying torque to implants within the body.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

Described herein are systems, apparatuses devices, and methods that facilitate the anchoring of a tissue anchor or similar device or structure to tissue, e.g., heart tissue of a subject (living subject, a simulation, etc.). For instance, some implementations provide improved techniques and devices for driving tissue anchors into the tissue of the heart, without requiring welding of portions of the driver to each other, in a manner that is reliable, predictable, and safe.

In accordance with some implementations herein an apparatus and/or system (which can be used with a valve of a heart, e.g., of a living subject or of a simulation), includes an implant, e.g., a tissue anchor, the tissue anchor having an anchor head and a tissue-engaging element.

In some implementations, the tissue engaging element can extend away from the anchor head to define an anchor axis of the tissue anchor. The anchor head can include an engagement surface, and, proximally from the engagement surface, one or more protrusions that extend medially.

In some implementations, the apparatus can further include a driver (e.g., an anchor driver and/or an implant driver) including a shaft and a drive head. The drive head is disposed at a distal end of the shaft. The drive head can define one or more engagement fingers, each of the engagement fingers having a root, and a tip distal from the root.

In some implementations, the drive head can be configured to engage the anchor head such that, for each of the engagement fingers, the engagement finger extends distally beyond the one or more protrusions of the anchor head, such that the tip is engaged with the engagement surface in a manner that inhibits axial movement of the drive head relative to the anchor head, and the root is disposed against a respective protrusion of the anchor head in a manner that delivers torque to the anchor head upon rotation of the shaft.

It is to be appreciated that while torque is delivered from the driver to the tissue anchor at the roots of the engagement fingers, engagement between the driver and the tissue anchor is retained by forces at the engagement fingers'tips. In some implementations, such separation between applied forces can reduce the risk of disengagement between the driver and the tissue anchor during driving of the tissue anchor into the tissue. In some implementations, the separation between applied forces can also increase the torque applied for driving of the tissue anchor, because the entire force at that location is used for a single action of driving the anchor.

In some implementations, the driver can be used to actuate an implant, and in such context the driver can be considered to be an implant driver. In some such implementations, the implant comprises an interface that is similar to the tissue anchor's anchor head. In this way, the implant driver can actuate the implant by transferring torque to the interface. For example, the implant can comprise an implant-modification mechanism that is actuated by receiving the torque at the interface, which changes a conformation (e.g., a shape or dimension) of the implant. In some such implementations, implant assumes an actuation-conformation, e.g., in which the implant engages tissue of the subject.

For purposes of summarizing the disclosure, certain aspects, advantages and novel features have been described. It is to be understood that not necessarily all such advantages may be achieved in accordance with any particular implementation. Thus, the disclosed implementations may be carried out in a manner that achieves or optimizes one advantage or group of advantages as taught herein without necessarily achieving other advantages as may be taught or suggested herein.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the techniques, methods, operations, steps, etc. described or suggested herein can be performed on a living animal (e.g., human, other mammal, etc.) or on a non-living simulation such as a cadaver, a cadaver heart, an anthropomorphic ghost, and/or a simulator device (which may include computerized and/or physical representations of body parts, tissue, etc.).

There is therefore provided, in accordance with some implementations, apparatus for use with a heart of a subject, the apparatus including a tissue anchor and/or a driver. The tissue anchor may have an anchor head and/or a tissue-engaging element that extends away from the anchor head to define an anchor axis of the tissue anchor.

The anchor head may have an engagement surface. Proximally from the engagement surface, the anchor head may have one or more protrusions that extend medially.

The driver may include a shaft and/or a drive head, disposed at a distal end of the shaft.

The drive head may define one or more engagement fingers, each of the engagement fingers having a root, and a tip distal from the root.

The drive head may be configured to engage the anchor head such that, for each of the engagement fingers, the engagement finger extends distally beyond the one or more protrusions of the anchor head. In some implementations, this engagement is such that the tip is engaged with the engagement surface in a manner that inhibits axial movement of the drive head relative to the anchor head, and/or the root is disposed against a respective protrusion of the anchor head in a manner that delivers torque to the anchor head upon rotation of the shaft.

For some implementations, at least one of the tissue anchor and the driver is sterile.

For some implementations, the driver defines two engagement fingers, the engagement fingers being parallel to each other and opposite each other.

For some implementations, the anchor head includes a wall circumscribing a cavity, the wall having a distal beveled surface forming the engagement surface.

For some implementations, the wall has a proximal beveled surface proximal to the distal beveled surface.

For some implementations, during insertion of the drive head into the anchor head, the tip of each of the engagement fingers deflects medially by the proximal beveled surface, and snaps radially to engage the distal beveled surface.

For some implementations, for removal of the driver from the tissue anchor, the tip of each of the engagement fingers deflects medially against the distal beveled surface until the tip disengages from the engagement surface.

For some implementations, the one or more protrusions are disposed at a proximal end of the anchor head.

For some implementations, the driver defines a lumen extending longitudinally through the shaft and through the drive head.

For some implementations, the one or more protrusions of the anchor head are substantially triangular and have a narrower portion disposed medially to a wider portion thereof.

For some implementations, a proximal surface of each of the one or more protrusions is substantially planar.

For some implementations, a proximal surface of each of the one or more protrusions includes a beveled portion, the beveled portion configured to assist in aligning the driver relative to the anchor head.

For some implementations, the driver includes a unitary elongate tube that defines the drive head and the shaft.

For some implementations, the one or more fingers of the driver are formed by laser cutting at least one slot into the unitary elongate tube.

For some implementations, the tube includes a plurality of cuts that configure the shaft as a flexible hypotube.

For some implementations, each of the engagement fingers includes a cut-out, proximal to the tip thereof, the cut-out being laser cut into the unitary elongate tube.

For some implementations, a connection between the shaft and the drive head of the driver is devoid of welding and of adhesives.

For some implementations, when no force is applied to the drive head, an external diameter of the drive head at the tips of the engagement fingers is not greater than an external diameter of the shaft.

For some implementations, the system further includes a locking rod received in a lumen extending through the driver, such that a presence of the locking rod within the drive head obstructs disengagement of the drive head from the anchor head.

For some implementations, the driver further includes: a spring, adapted to push the locking rod toward being disposed within the drive head; a detent, at a distal part of the driver, adapted to obstruct the locking rod from being pushed into the drive head; and/or a mount on which the detent is mounted, the mount: coupled to the shaft, having a leading surface, and/or adapted such that advancing the drive head into engagement with the anchor head slides the leading surface over the anchor head in a manner that withdraws the detent from obstructing the locking rod.

For some implementations, a surface of the anchor head, distal to the tissue-engaging element, has beveled wall section, and the leading surface of the mount is adapted to slide over the beveled wall section.

For some implementations, the leading surface of the mount is a beveled surface.

For some implementations, the mount has a blocking operative state, in which the detent obstructs the locking rod from being pushed into the drive head, and an open operative state, in which the detent is withdrawn from obstructing the locking rod.

For some implementations, sliding of the leading surface of the mount over the anchor head causes portions of the mount, onto which the detent is mounted, to pivot radially outwardly, to withdraw the detent from obstructing the locking rod.

For some implementations, the detent includes at least one protrusion, received in a slot at the distal part of the driver, such that the protrusions are disposed within the lumen and obstruct the locking rod.

For some implementations, the system further includes a funnel, having a cylindrical exterior surface and a beveled interior surface, the funnel being attachable to a proximal portion of the anchor head and being configured to receive the driver and to guide the driver to the anchor head, for engagement therebetween.

There is further provided, in accordance with some implementations, an apparatus, including a tissue anchor and/or a driver. The tissue anchor may include a tissue-engaging element and/or an anchor head.

The driver may include a shaft defining a lumen, and/or a drive head fixed to a distal end of the shaft. The driver may be configured to reversibly engage the anchor head.

The apparatus may include a locking rod that may extend from a proximal part of the driver through the lumen, and may be adapted such that a presence of the locking rod within the drive head obstructs disengagement of the drive head from the anchor head, and/or a spring, adapted to push the locking rod toward being disposed within the drive head.

The driver may define (i) a detent, at a distal part of the driver, that may be adapted to obstruct the locking rod from being pushed into the drive head; and/or (ii) a mount on which the detent may be mounted. The mount may be coupled to the shaft, and may have a leading surface, and/or be adapted such that advancing the drive head into engagement with the anchor head slides the leading surface over the anchor head in a manner that withdraws the detent from obstructing the locking rod.

For some implementations, at least one of the tissue anchor and the driver is sterile.

For some implementations, a surface of the anchor head, distal to the tissue-engaging element, has beveled wall section, and the leading surface of the mount is adapted to slide over the beveled wall section.

For some implementations, the leading surface of the mount is a beveled surface.

For some implementations, the mount has a blocking operative state, in which the detent obstructs the locking rod from being pushed into the drive head, and an open operative state, in which the detent is withdrawn from obstructing the locking rod.

For some implementations, sliding of the leading surface of the mount over the anchor head causes portions of the mount, onto which the detent is mounted, to pivot radially outwardly, to withdraw the detent from obstructing the locking rod.

For some implementations, the detent includes one or more protrusion, received in a slot at the distal part of the driver, such that each protrusion is disposed within the lumen and obstructs the locking rod.

For some implementations, the tissue-engaging element extends away from the anchor head to define an anchor axis of the tissue anchor, and the anchor head has an engagement surface, and, proximally from the engagement surface, the one or more protrusions extend medially.

For some implementations, the drive head of the driver is disposed at a distal end of the shaft, defines one or more engagement fingers, each of the engagement fingers having a root, and a tip distal from the root, and is configured to engage the anchor head, wherein, for each of the engagement fingers, the engagement finger extends distally beyond the one or more protrusions of the anchor head, such that: the tip is engaged with the engagement surface in a manner that inhibits axial movement of the drive head relative to the anchor head, and/or the root is disposed against a respective protrusion of the anchor head in a manner that delivers torque to the anchor head upon rotation of the shaft.

For some implementations, the driver defines two engagement fingers, the engagement fingers being parallel to each other and opposite each other.

For some implementations, the anchor head includes a wall circumscribing a cavity, the wall having a distal beveled surface forming the engagement surface.

For some implementations, the wall has a proximal beveled surface proximal to the distal beveled surface.

For some implementations, during insertion of the drive head into the anchor head, the tip of each of the engagement fingers deflects medially by the proximal beveled surface, and snaps radially to engage the distal beveled surface.

For some implementations, for removal of the driver from the tissue anchor, the tip of each of the engagement fingers deflects medially against the distal beveled surface until each of the tips disengage from the engagement surface.

For some implementations, the one or more engagement fingers of the driver are formed by laser cutting at least one slot into a unitary elongate tube.

For some implementations, each of the engagement fingers includes a cut-out, proximal to the tip thereof, the cut-out being laser cut into the unitary elongate tube.

For some implementations, when no force is applied to the drive head, an external diameter of the drive head at the tips of each of the engagement fingers is not greater than an external diameter of the shaft.

For some implementations, the one or more protrusions are disposed at a proximal end of the anchor head.

For some implementations, the one or more protrusions are substantially triangular and have a narrower portion disposed medially to a wider portion thereof.

For some implementations, a proximal surface of each of the protrusions is substantially planar.

For some implementations, a proximal surface of each of the protrusions has at least a beveled portion, the beveled portion configured to assist in aligning the driver relative to the anchor head.

For some implementations, the shaft includes a plurality of cuts that configure the shaft as a flexible hypotube.

For some implementations, the drive head and the shaft are formed of a unitary elongate tube, and the shaft is configured to transluminally advance the drive head, engaged with the tissue anchor, to a heart of a subject while a proximal part of the shaft remains outside of the subject.

For some implementations, a connection between the shaft and the drive head of the driver is devoid of welding and of adhesives.

For some implementations, the system further includes a funnel, having a cylindrical exterior surface and a beveled interior surface, the funnel being attachable to a proximal portion of the anchor head and being configured to receive the driver and to guide the driver to the center of the anchor head, for engagement therebetween.

There is further provided, in accordance with some implementations, apparatus for use with a heart of a subject, the apparatus including a driver adapted to engage an anchor head of a tissue anchor. The driver may include a unitary elongate tube that defines: a drive head, that may define at least three longitudinal fingers separated by at least three longitudinal slots. The longitudinal fingers may be configured to engage the anchor head by each of the longitudinal fingers engaging with a respective portion of the anchor head.

The driver may include a shaft, which may be configured to transluminally advance the drive head, engaged with the tissue anchor, to the heart while a proximal part of the shaft remains outside of the subject.

For some implementations, the apparatus further includes the tissue anchor.

For some implementations, at least one of the tissue anchor and the driver is sterile.

For some implementations, the system further includes a locking rod received in a lumen of the shaft to prevent detachment of the driver from the anchor head.

For some implementations, the anchor head defines a cavity having at least three lobes, and each of the at least three longitudinal fingers of the driver engages with one of the at three lobes.

For some implementations, the at least three longitudinal slots of the driver are laser cut into the unitary elongate tube to form the drive head.

For some implementations, a distal end of each of the at least three longitudinal fingers of the driver includes a radially inward facing tooth.

For some implementations, the shaft and the drive head of the driver are connected, the connection between the shaft and the drive head being devoid of welding and of adhesives.

For some implementations, the tube includes a plurality of cuts that configure the shaft as a flexible hypotube.

For some implementations, when no force is applied to the drive head of the driver, an external diameter of the drive head is equal to, or smaller than, an external diameter of the shaft.

For some implementations, upon insertion of the drive head into the anchor head of the tissue anchor the drive head remains disengaged from the anchor head, and upon insertion of the locking rod into the drive head the longitudinal fingers of the drive head are pushed radially outwardly to engage the anchor head.

For some implementations, removal of the locking rod from the lumen of the driver causes the longitudinal fingers to disengage from the anchor head.

For some implementations, when no force is applied to the drive head of the driver, an external diameter of at least a distal end the drive head is greater than an external diameter of the shaft.

For some implementations, when the driver is disposed within the anchor head, the longitudinal fingers of the driver snap fit into a cavity in the anchor head for engagement therebetween, and the locking rod prevents medial motion of the longitudinal fingers, thereby preventing disengagement of the longitudinal fingers from the anchor head.

For some implementations, following removal of the locking rod from the lumen of the driver, the longitudinal fingers can disengage from the anchor head by deflecting medially against beveled surface of the anchor head.

There is further provided, in accordance with some implementations, a method of manufacture of a driver. The method may include cutting at least three longitudinal slots in one end of an elongate tube. The cutting may define at least three longitudinal fingers and/or form a plurality of cuts in the remainder of the elongate tube.

The cutting may configure the remainder of the elongate tube as a flexible hypotube. The end of the elongate tube may include the at least three longitudinal slots including a drive head or the driver, and/or the remainder of the elongate tube may include a shaft of the driver.

For some implementations, the method further includes bending a tip of each of the at least three longitudinal fingers to form a point that extends medially.

For some implementations, cutting of the at least three longitudinal slots and/or forming of the plurality of cuts includes laser cutting the elongate tube.

For some implementations, the method further includes deflecting an end of each of the at least three fingers in a radial direction, such that a circumference of the driver at the drive head is greater than a circumference of the shaft.

For some implementations, the method is devoid of welding and of adhering.

For some implementations, the method further includes sterilizing the driver.

There is further provided, in accordance with some implementations, apparatus for use with a heart of a subject, the apparatus including a driver. The driver may include a shaft and/or a drive head disposed at a distal end of the shaft. The drive head may include at least two fingers and at least one slot disposed between the at least two fingers.

The driver may be configured to mate with an anchor head of a tissue anchor. The mating may be such that during insertion of the driver into the anchor head, the at least two fingers may be deflected laterally in a manner that may inhibit axial movement of the drive head relative to the anchor. The mating may be such that at least one slot may abut a protrusion of the anchor head in a manner that may deliver torque to the anchor head upon rotation of the shaft.

For some implementations, the driver is sterile.

There is further provided, in accordance with some implementations, apparatus for use with a tissue of a subject, the apparatus including an implant and an implant driver.

The implant may have an implant head and an implant body that may extend away from the implant head. The implant head may have an engagement surface, and/or proximally from the engagement surface, one or more protrusions that extend medially.

The implant driver may include a shaft and/or a drive head. The drive head may be disposed at a distal end of the shaft.

The drive head may define one or more engagement fingers, each of the engagement fingers having a root, and a tip distal from the root, and/or configured to engage the implant head. The engagement may be such that, for each of the engagement fingers, the engagement finger extends distally beyond the one or more protrusions of the implant head. The engagement finger may extend distally such that: the tip is engaged with the engagement surface in a manner that may inhibit axial movement of the drive head relative to the implant head, and/or the root is disposed against a respective protrusion of the implant head in a manner that may transfer torque to the implant head upon rotation of the shaft.

For some implementations, at least one of the implant and the implant driver is sterile.

For some implementations, the implant is configured such that transfer of torque to the implant head actuates the implant.

For some implementations, the implant is configured such that transfer of torque to the implant head modifies a conformation of the implant into an actuation conformation.

For some implementations, the implant is configured to retain the actuation conformation following transfer of torque to the implant head.

For some implementations: the implant driver is configured to, following transfer of the torque to the implant head, disengage the implant head, and/or the implant is configured to retain the actuation conformation following disengagement of the implant driver from the implant head.

There is further provided, in accordance with some implementations, apparatus for use with a subject, the apparatus including a delivery tool and an implant, configured to be delivered into the subject.

The implant may include an implant-modification mechanism (IMM). The IMM may be configured to, upon actuation thereof, modify a conformation of the implant.

The IMM may define an interface, operatively coupled to the IMM such that torque applied to the interface may actuate the IMM. The IMM may have an engagement surface, and/or, proximally from the engagement surface, one or more protrusions that extend medially.

The delivery tool may be configured to deliver the implant into the subject, and may include a driver. The driver may include a shaft and a drive head.

The drive head may be disposed at a distal end of the shaft, and may define one or more engagement fingers. Each of the engagement fingers may have a root, and a tip distal from the root. Each of the engagement fingers may be configured to engage the interface such that, for each of the engagement fingers, the engagement finger extends distally beyond the one or more protrusions of the interface.

For each of the engagement fingers, the tip may be engaged with the engagement surface in a manner that may inhibit axial movement of the drive head relative to the interface, and/or the root may be disposed against a respective protrusion of the interface in a manner that may deliver the torque to the interface upon rotation of the shaft.

For some implementations, at least one of the implant and the delivery tool is sterile.

The present invention will be more fully understood from the following detailed description of applications thereof, taken together with the drawings, in which:

### BRIEF DESCRIPTION OF THE FIGURES

The foregoing discussion will be understood more readily from the following detailed description when taken in conjunction with the accompanying Figures, in which:

FIGS. 1A, 1B, and 1C are perspective view illustrations of an apparatus and its aesthetic features, the apparatus including a tissue anchor and a driver, at different stages of connection therebetween, in accordance with some implementations;

FIGS. 2A, 2B, and 2C are sectional illustrations of the apparatus of FIGS. 1A to 1C at corresponding stages of connection, in accordance with some implementations;

FIGS. 3A, 3B, and 3C are perspective view illustrations of an apparatus and its aesthetic features, the apparatus including a tissue anchor and a driver, at different stages of connection therebetween, in accordance with some implementations;

FIGS. 4A, 4B, 4C, and 4D are sectional illustrations of an apparatus including a tissue anchor and a driver, at different stages of connection therebetween, in accordance with some implementations;

FIGS. 5A, 5B, 5C, and 5D are perspective view illustrations of an apparatus and its aesthetic features, the apparatus including a tissue anchor and a driver, at different stages of connection therebetween, in accordance with some implementations;

FIGS. 6A, 6B, and 6C are sectional illustrations of the apparatus of FIGS. 5A, 5C, and 5D at corresponding stages of connection, in accordance with some implementations;

FIG. 7 is a perspective view illustration and a sectional illustration of an apparatus and its aesthetic features, the apparatus including a tissue anchor and a driver, in accordance with some implementations;

FIGS. 8A and 8B are, respectively, a perspective view illustration and a sectional illustration of a mount and its aesthetic features, the mount usable and/or suitable for use with the apparatus of FIGS. 5A to 7, in accordance with some implementations;

FIGS. 9A, 9B, and 9C are perspective view illustrations of an apparatus and its aesthetic features, the apparatus including a tissue anchor and a driver, at different stages of connection therebetween, in accordance with some implementations;

FIGS. 10A, 10B, and 10C are sectional illustrations of the apparatus of FIGS. 9A to 9C at corresponding stages of connection, in accordance with some implementations;

FIGS. 11A, 11B, and 11C are sectional illustrations of an apparatus and its aesthetic features, the apparatus similar to that of FIGS. 9A to 9C at corresponding stages of connection, in accordance with some implementations; and

FIG. 12A-B and 13A-B are schematic illustrations showing apparatuses comprising an implant driver and an implant, in accordance with some implementations.

### DETAILED DESCRIPTION

The principles of the tissue anchors may be better understood with reference to the drawings and the following description.

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features can be omitted or simplified in order not to obscure the disclosure. Additionally, in order to avoid undue clutter from having too many reference numbers and lead lines on a particular drawing, some elements may not be explicitly identified in every drawing that contains that element.

The present disclosure includes different variants of some elements. Variants of a given element typically have the same structure and/or function as each other except for any differences described. For any given element for which different variants are disclosed, the identical name is used for each variant, in order to denote that they are, in fact, variants the same given element. Unless stated otherwise, applications of the devices, systems, and techniques described herein may include any arrangement in which one variant of an element is substituted with another identically-named variant of that element. Furthermore, throughout the figures, suffixes are used to denote different variants of the same element. Unless stated otherwise, such variants may be substituted with each other, mutatis mutandis. That is, unless stated otherwise, any element having a given reference numeral may be substituted with any other element (e.g., any other variant of the element) having the same reference numeral, independent of any suffix.

In order to avoid undue clutter from having too many reference numbers and lead lines on a particular drawing, some elements are introduced via one or more drawings and not explicitly identified in every other drawing that contains that element.

It is to be understood that the scope of the disclosure is not limited in its implementation to the details of construction and the arrangement of the components set forth in the following description or illustrated in the drawings. The disclosure is capable of other implementations or of being practiced or carried out in various ways. Furthermore, it is to be understood that the phraseology and terminology employed in the disclosure is for the purpose of description and should not be regarded as limiting.

For the purposes of this implementation, the term “subject” relates to any mammal, particularly humans.

For the purposes of this implementation, the term “cardiac tissue” relates to any tissue of the heart, and includes, for example, any wall of the heart and any tissue of any heart valve.

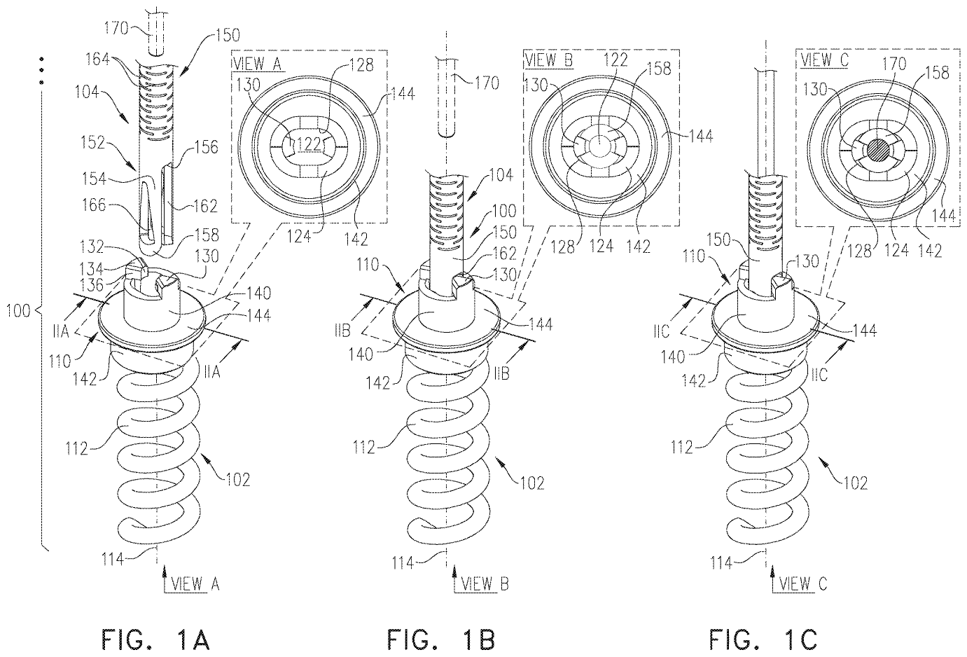

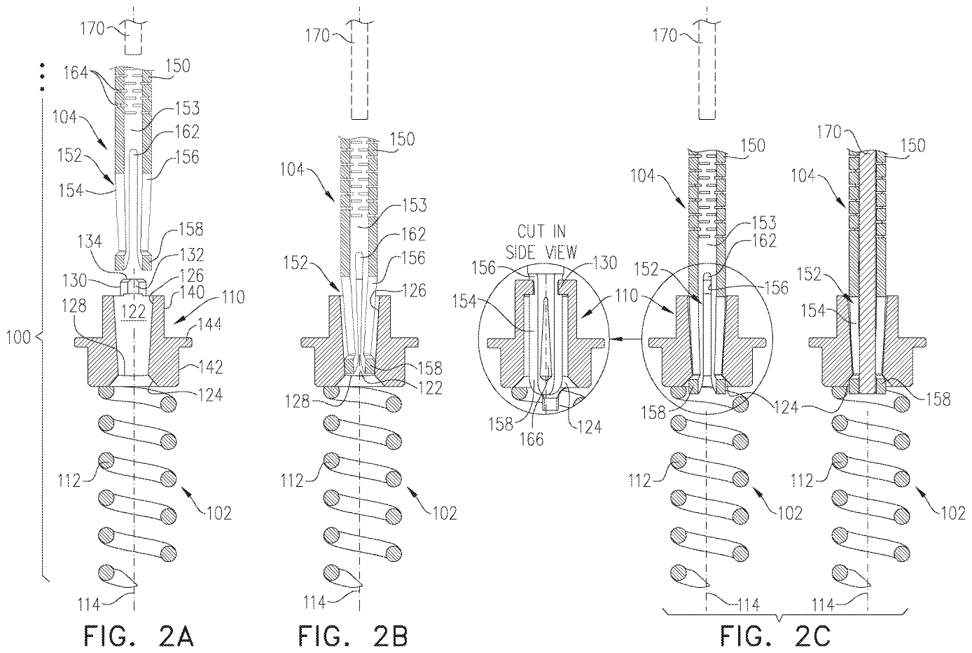

Referring now to the drawings, an apparatus 100 usable and/or configured for use in the heart of a subject (e.g., a living subject, a simulation, etc.) is shown in FIGS. 1A to 1C in a perspective view, and in FIGS. 2A to 2C in sectional views, according to some implementations. Apparatus 100 includes a tissue anchor 102 and a driver 104, shown in different stages of connection therebetween. The sectional illustrations of FIGS. 2A, 2B, and 2C, are taken along corresponding section lines IIA-IIA, IIB-IIB, and IIC-IIC, in FIGS. 1A, 1B, and 1C, respectively.

As seen in FIGS. 1A and 2A, tissue anchor 102 includes an anchor head 110 and a helical tissue-engaging element 112, which extends away from anchor head 110 to define an anchor axis 114. Anchor head 110 includes an inner wall circumscribing a cavity 122. A distal end of the inner wall is beveled, and defines an engagement surface 124. In some implementations, the inner wall defines a proximal beveled surface 126 (FIG. 2A-C), which converges with engagement surface 124 at a ridge 128.

In some implementations, an angle between proximal beveled surface 126 and anchor axis 114, is smaller than an angle between engagement surface 124 and anchor axis 114.

Proximally to engagement surface 124, anchor head 110 includes one or more protrusions 130, that extend medially. In some implementations, and as illustrated, two protrusions 130 are disposed at a proximal end of proximal beveled surface 126 and of anchor head 110. The protrusions 130 extend medially across a portion of cavity 122 (e.g., extend medially from an outer perimeter of the anchor head 110), as shown in view A of FIG. 1A, which is a planar bottom view of the apparatus.

In some implementations, protrusions 130 are substantially triangular, and have a narrower portion disposed medially to a wider portion thereof. In some implementations, protrusions 130 include a medial surface 132 (e.g., parallel and/or coaxial to the anchor axis 114), a proximal surface 134 (e.g., such that the proximal surface is positioned on a transverse plane associated with the anchor axis 114), and side walls 136 (e.g., such that a sidewall is positioned on a longitudinal plane associated with the anchor axis). In some implementations, and as shown in FIGS. 1A to 2C, proximal surface 134 is planar.

As explained in further detail hereinbelow, one or more of cavity 122, protrusions 130, and engagement surface 124 together form a driving interface for engagement with driver 104.

In some implementations, an outer surface of anchor head 110 may include a proximal portion 140 having a first circumference, and a distal portion 142 having a second circumference. In some implementations, the second circumference can be greater than the first circumference, such that a shoulder is formed between proximal portion 140 and distal portion 142. In some implementations, and as illustrated, a flange 144 can extend radially out of the outer surface of anchor head 110, at the shoulder between proximal portion 140 and distal portion 142.

Driver 104 includes a shaft 150 and a drive head 152 disposed at a distal end of the shaft. A lumen 153 extends longitudinally through shaft 150 and through drive head 152. Drive head 152 defines one or more engagement fingers 154, here illustrated as two engagement fingers. Each engagement finger 154 defines a root 156 and a tip 158, distal from the root. In some implementations, tips 158 of engagement fingers 154 are curved, and/or have a triangular contour, as shown in FIG. 1A. In some implementations, and as illustrated, engagement fingers 154 are parallel to each other (e.g., side walls thereof are parallel to each other), and are opposite each other (e.g., disposed on opposing sides of a central longitudinal axis of driver 104). In some implementations, and as illustrated, drive head 152 of driver 104 is side-to-side symmetrical.

In some implementations, drive head 152 may include more than two engagement fingers, e.g., three engagement fingers, four engagement fingers, five engagement fingers, etc. In some implementations, the drive head may not be side-to-side symmetrical.

In some implementations, driver 104 comprises a unitary elongate tube that defines shaft 150 and drive head 152, such that a connection between the shaft and the drive head is devoid of welding and of adhesives. In some implementations, engagement fingers 154 are formed by laser cutting slots 162, into the unitary elongate tube, thereby separating the fingers from each other. In some implementations, the unitary elongate tube further includes a plurality of cuts 164, that configure shaft 150 as a flexible hypotube.

In some implementations, one or more of engagement fingers 154 can define a cut-out 166 therein, proximal to tip 158. In some implementations, cut-out 166 is laser cut into the unitary elongate tube. In some implementations, and as shown in FIG. 1A, when no forces are applied to drive head 152, an external diameter of the drive head at tips 158 of engagement fingers 154 is not greater than an external diameter of shaft 150.

It is to be appreciated that torque is delivered from driver 104 to tissue anchor 102 at a first location along anchor axis 114, namely at protrusions 130, while engagement between the driver and the tissue anchor is retained by forces applied at a second location along anchor axis 114, namely at tips 158 of fingers 154. In some implementations, such separation between applied forces can reduce the risk of disengagement between the driver and the tissue anchor during driving of the tissue anchor into the tissue. In some implementations, the separation between applied forces can also increase the torque applied for driving of the tissue anchor, because the entire force at that location is used for a single action of driving the anchor.

FIGS. 1A and 2A illustrate apparatus 100 with driver 104 longitudinally aligned with and disposed proximally to tissue anchor 102, and ready for engagement therebetween, in accordance with some implementations. Alternatively, apparatus 100 can be provided with driver 104 (e.g., drive head 152 thereof) already engaged to tissue anchor 102. As seen, slots 162 of driver 104 are vertically aligned with protrusions 130, such that the protrusions can slide into the slots and engagement fingers 154 can fit into cavity 122 between protrusions 130.

FIGS. 1B and 2B illustrate driver 104 having being advanced distally to engage anchor head 110, in accordance with some implementations. In the state shown in FIGS. 1B and 2B, a distal end of drive head 152 is disposed within cavity 122. Tips 158 of engagement fingers 154 are deflected medially against proximal beveled surface 126 (FIG. 2B), while protrusions 130 are disposed within slots 162 (FIG. 1B).

FIGS. 1C and 2C illustrate apparatus 100 where driver 104 is engaged with tissue anchor 102, in accordance with some implementations. As shown in the transition between FIGS. 2B and 2C, tips 158 of engagement fingers 154 have snapped radially (e.g., radially outward) to engage engagement surface 124. This engagement inhibits axial movement of drive head 152 relative to tissue anchor 102.

In some implementations, and as shown in the transition between the left and right frames of FIG. 2C, a longitudinal locking rod 170 is received within lumen 153 extending through driver 104. Presence of locking rod 170 within lumen 153 obstructs disengagement of drive head 152 from anchor head 110, by preventing medial deflection of tips 158 to move past ridge 128. In some implementations, the presence of the locking rod 170 further inhibits axial movement between the anchor head 110 and drive head 152 by ensuring that the tips 158 of the engagement fingers 154 remain engaged with and/or mated with the engagement surface 124 of the anchor head.

In accordance with some implementations, and as shown in FIG. 1C, roots 156 of engagement fingers 154 are disposed against side walls 136 of protrusions 130, e.g., such that the protrusions 130 are disposed in and/or mated with the slots 162 between the engagement fingers 154. As such, when shaft 150 of driver 104 is rotated, torque is delivered from drive head 152 (e.g., from roots 156 of engagement fingers 154) to anchor head 110 (e.g., to protrusions 130).

In some implementations, for removal of driver 104 from tissue anchor 102, locking rod 170 is first removed from lumen 153 (not shown). Subsequently, as shaft 150 is pulled in a proximal direction, engagement fingers 154 deflect medially against engagement surface 124 until tips 158 of the engagement fingers disengage from the engagement surface, and pass ridge 128. The fingers can then deflect radially against proximal beveled surface 126 until drive head 152 is removed from cavity 122 of anchor head 110.

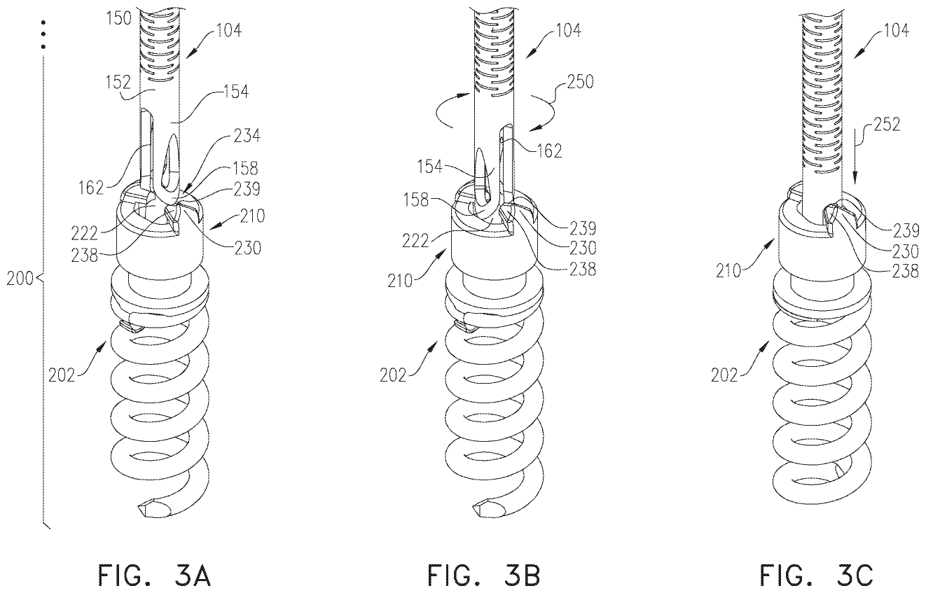

Reference is now made to FIGS. 3A, 3B, and 3C, which are perspective view illustrations of an apparatus 200 including a tissue anchor 202 and driver 104, at different stages of connection therebetween, in accordance with some implementations. Driver 104 is as described hereinabove with respect to FIGS. 1A to 2C.

Tissue anchor 202 is substantially similar to tissue anchor 102 described hereinabove with respect to FIGS. 1A to 2C, with like reference numerals indicating like elements. However, in tissue anchor 202, proximal surfaces 234 of protrusions 230 include at least one beveled portion 238. In some implementations, and as illustrated, proximal surfaces 234 are substantially triangular, and include two beveled portions 238 converging at a ridge 239.

FIG. 3A illustrates apparatus 200 with driver 104 longitudinally aligned with and disposed proximally to tissue anchor 202. Alternatively, apparatus 200 can be provided with driver 104 (e.g., drive head 152 thereof) already engaged to tissue anchor 202.

In some implementations, and as shown, the driver is not properly rotationally aligned with the tissue anchor, since the fingers'tips 158 are vertically aligned with protrusions 230. As such, protrusions 230 are not aligned to slide into slots 162 and fingers 154 are not aligned to slide into cavity 222, between protrusions 230, when the driver is advanced distally.

In some implementations, when the driver 104 is advanced toward tissue anchor 202, beveled portions 238 of protrusions 230 are engaged by tips 158 of driver 104. The beveled portions function as cams, causing rotation of driver 104 in the direction indicated by arrows 250 shown in FIG. 3B. This assists in alignment of driver 104 into the position shown in FIG. 3B, in which protrusions 230 are vertically aligned with slots 162, and engagement fingers 154 are vertically aligned with cavity 222 between protrusions 230. Once in the position of FIG. 3B, driver 104 can advanced further (e.g., distally), in the direction of arrow 252 shown in FIG. 3C, substantially as described hereinabove with respect to FIGS. 1B and 2B.

FIG. 3C illustrates driver 104 engaged with tissue anchor 202, in a state substantially described hereinabove with respect to FIGS. 1C and 2C. In this position, locking rod 170 may be advanced into drive head 152 of driver 104 to prevent detachment of the driver from tissue anchor 202, substantially as described hereinabove. Once engaged, torque can be delivered from driver 104 to tissue anchor 202, substantially as described hereinabove with respect to FIGS. 1C and 2C.

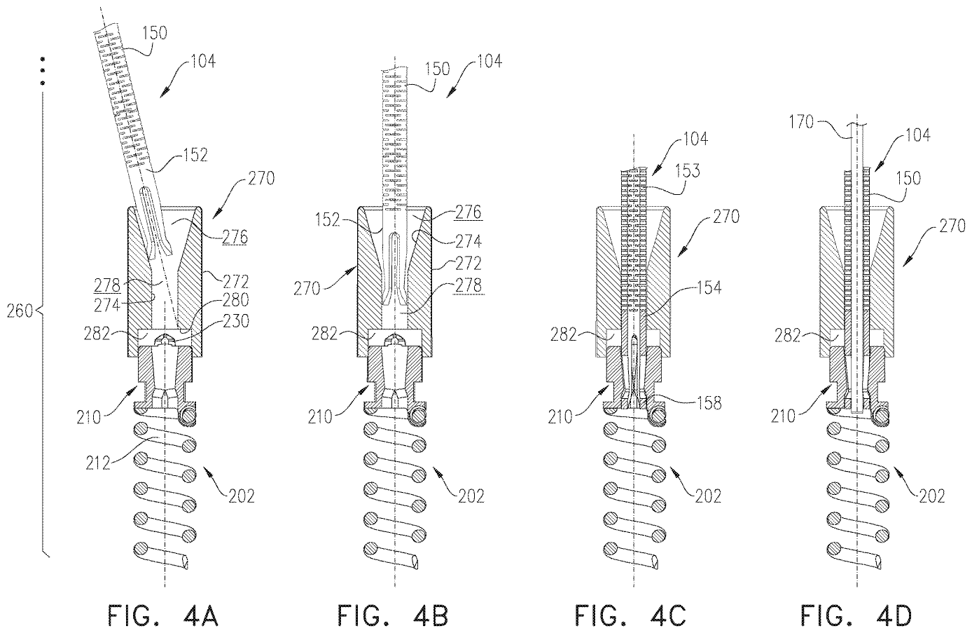

Reference is now made to FIGS. 4A, 4B, 4C, and 4D, which are sectional illustrations of an apparatus 260 including tissue anchor 202 and driver 104, at different stages of connection therebetween, in accordance with some implementations.

Apparatus 260 is substantially similar to apparatus 200 described hereinabove with respect to FIGS. 3A to 3C, and includes driver 104, tissue anchor 202, and locking rod 170 as described. Apparatus 260 additionally includes a funnel 270, attachable to a proximal portion of anchor head 210.

In some implementations, and as shown in at least FIG. 4A, funnel 270 has a generally cylindrical exterior surface 272, and defines an internal wall portion 274 circumscribing a cavity. The cavity includes a proximal beveled portion 276 leading to a central tubular portion 278. Near a distal end of funnel 270, the cavity has a greater diameter than in central tubular portion 278, and forms an anchor head seat 282, such that a shoulder 280 is defined between the central tubular portion 278 and the anchor-head seat 282. Anchor-head seat 282 is sized and configured to accommodate a proximal end of anchor head 210 of anchor 202.

FIG. 4A illustrates anchor head 210 of tissue anchor 202 disposed within anchor-head seat 282 of funnel 270, in accordance with some implementations. Drive head 152 of driver 104 is inserted into the cavity of the funnel, and slides distally through proximal beveled portion 276 toward central tubular portion 278. In this way, drive head 152 is guided toward the central tubular portion 278 by wall portion 274 surrounding proximal beveled portion 276.

In FIG. 4B, drive head 152 has reached central tubular portion 278 of funnel 270, and is now longitudinally aligned with tissue anchor 202 for engagement therebetween, in accordance with some implementations. Engagement between tissue anchor 202 and driver 104 can be substantially as described hereinabove with respect to FIGS. 1A to 2C or with respect to FIGS. 3A to 3C.

FIG. 4C illustrates driver 104 fully engaged with anchor head 210, such that tips 158 of engagement fingers 154 contact engagement surface 224, in accordance with some implementations. In FIG. 4D, locking rod 170 has been advanced into lumen 153 of driver 104 to prevent axial movement of drive head 152 relative to tissue anchor 202, in accordance with some implementations.

It is to be appreciated that although the apparatus of FIGS. 4A to 4D is illustrated using tissue anchor 202 of FIGS. 3A to 3C, it can be implemented in a similar manner using tissue anchor 102 described hereinabove with respect to FIGS. 1A to 2C.

In some implementations, shaft 150 of driver 104 is configured to transluminally advance drive head 152 to the heart of a subject (e.g., a living subject, a simulation, etc.), while engaged with tissue anchor 202 and such that a proximal part of the shaft remains outside of the subject's body. It is to be appreciated that, in some implementations, when driver 104 advances tissue anchor 202 into the body of the subject, tissue anchor 202 can be pushed out of anchor-head seat 282 and advanced distally, such that funnel 270 remains disposed about shaft 150 of the driver until the driver is retracted from the body of the subject.

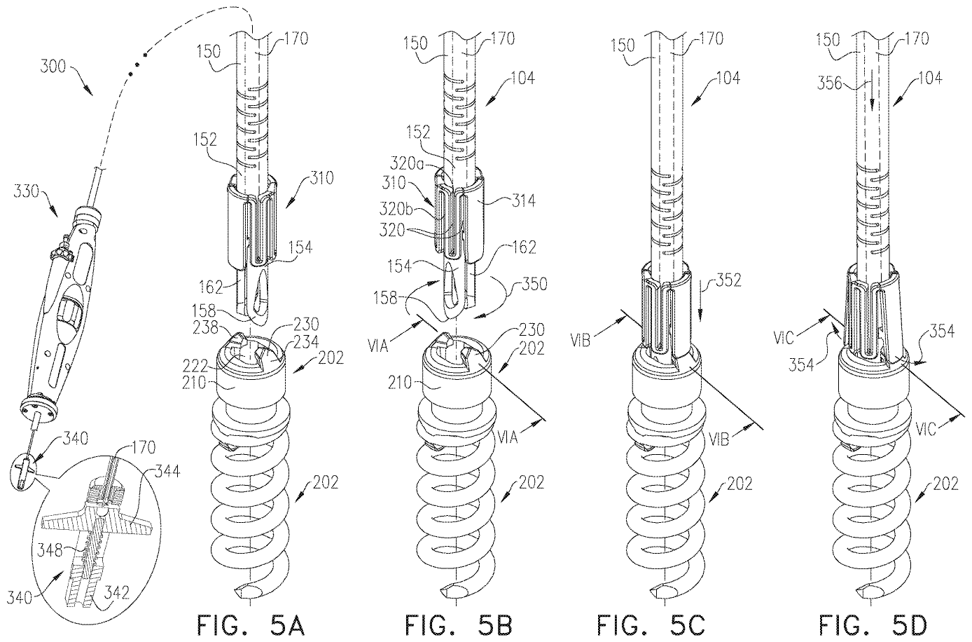

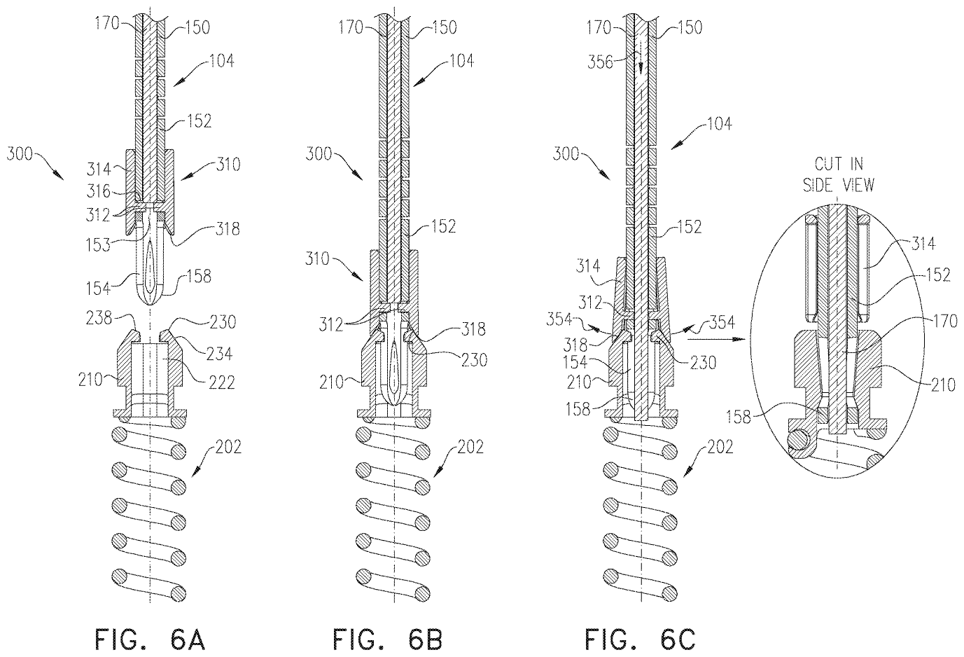

Reference is now made to FIGS. 5A, 5B, 5C, and 5D, which are perspective view illustrations of an apparatus 300 in accordance with some implementations. Reference is additionally made to FIGS. 6A, 6B, and 6C, which are sectional illustrations of apparatus 300, taken along corresponding section lines VIA-VIA, VIB-VIB, and VIC-VIC, in FIGS. 5B, 5C, and 5D, respectively.

Apparatus 300 is substantially similar to apparatus 200 described hereinabove with respect to FIGS. 3A to 3C, and includes driver 104, tissue anchor 202, and locking rod 170 as described. Apparatus 300 is designed to automate insertion of rod 170 into lumen 153 within anchor head 210, as explained in further detail hereinbelow.

As seen in FIGS. 5A and 6A, apparatus 300 includes a mount 310, coupled to shaft 150 and disposed therearound. Mount 310 includes detent protrusions 312 which extend medially from a cylindrical body 314 of mount 310, and are adapted to be received within dedicated slots 316 at a distal portion of shaft 150. Cylindrical body 314 of mount 310 defines a leading surface 318 at a distal end thereof (FIG. 6A-C). In some implementations, and as shown, leading surface 318 can be beveled.

In some implementations, cylindrical body 314 of mount 310 has a plurality of longitudinal slots 320 (FIG. 5A-D) formed therein, which allow for some flexibility of mount 310, particularly for expansion and contraction in a radial direction relative to shaft 150. In some implementations and as shown in FIGS. 5A-5D, some of longitudinal slots 320, such as slot 320a, extend from a proximal end 322 toward a distal end 324 of cylindrical body 314. Other longitudinal slots 320, such as slot 420b, extend from distal end 324 of the cylindrical body toward proximal end 322. In some such implementations, and as shown, longitudinal slots 320 are formed in a portion of cylindrical body 314 which does not include detent protrusions 312.

In some implementations, and as shown in FIG. 5A, shaft 150 of driver 104 can extend within a channel, to a proximal end of a handle 330 of the apparatus 300. A proximal end of locking rod 170 can be coupled to an anchor release mechanism 340, e.g., as shown in the inset of FIG. 5A. In some implementations, the anchor release mechanism 340 can be positioned at a proximal end of handle 330. In such implementations, a proximal end of locking rod 170 can be coupled to a component of the anchor release mechanism at a proximal end of apparatus 300, e.g., at handle 330.

In some implementations, release mechanism 340 comprises a housing 342 and a finger-engager 344 that can be coupled to the proximal end of rod 170. In some such implementations, finger-engager 344 is coupled to housing 342 via a spring 348. For example, spring 348 can be adapted to push locking rod 170 distally, toward being disposed within drive head 152. A proximal end of shaft 150 of driver 104 is coupled to housing 342.

In some implementations, handle 330 is adapted to control operation of driver 104 (e.g., rotation and/or proximal-distal movement thereof, and/or release of tissue anchor 202). For some implementations, driver 104 can be electronically controllable, such as by using an extracorporeal controller and/or electric motor coupled to, or forming part of, handle 330 and/or housing 342.

In the state shown in FIGS. 5A and 6A, the mount's detent protrusions 312 are disposed within slots 316 of shaft 150, and extend medially into lumen 153 (visible in FIG. 6A). As such, detent protrusions 312 obstruct locking rod 170, which in some implementations, can be biased in a distal direction toward drive head 152, from being pushed into the drive head. Consequently, this arrangement of mount 310 may be considered a blocking operative state of the mount.

Additionally, in FIGS. 5A and 5B, driver 104 is illustrated as being longitudinally aligned with and disposed proximally to tissue anchor 202. Alternatively, apparatus 300 can be provided with driver 104 (e.g., drive head 152 thereof) already engaged to tissue anchor 202.

In some implementations, and as shown in FIG. 5A, the driver is not properly rotationally aligned with the tissue anchor, since fingers 154 of the driver are vertically aligned with protrusions 230. As such, protrusions 230 are not aligned to slide into slots 162 and fingers 154 are not aligned to slide into cavity 222, between protrusions 230, when the driver is advanced in a distal direction.

In some implementations, when the driver 104 is advanced toward tissue anchor 202, beveled portions 238 of protrusions 230 are engaged by tips 158 of driver 104. The beveled portions function as cams, causing rotation of driver 104 in the direction indicated by arrows 350 shown in FIG. 5B. This assists in alignment of driver 104 to be in the position shown in FIG. 5B, in which protrusions 230 are vertically aligned with slots 162, and engagement fingers 154 are vertically aligned with cavity 222 between protrusions 230.

FIGS. 5C and 6B illustrate apparatus 300 where driver 104 continues to be advanced in a distal direction shown by arrow 352 (FIG. 5C), such that engagement fingers 154 of the driver are disposed within cavity 222, and protrusions 230 are disposed within slots 162, as described hereinabove. As shown, leading surface 318 of mount 310 engages proximal surface 234 of protrusions 230.

In some implementations, as driver 104 advances further in a distal direction, leading surface 318 of mount 310 slides over a proximal surface of anchor head 210 (e.g., over proximal surface 234 of protrusions 230), causing at least a portion of mount 310 to deflect or pivot radially outwardly in the direction of arrows 354, as shown in FIGS. 5D and 6C. Deflection of mount 310 causes detent protrusions 312 to move (e.g., pivot) radially outwardly (FIG. 6C), in a manner that withdraws detent protrusions 312 from obstructing locking rod 170, such that the mount is in an open operative state.

In some implementations, and as shown in FIG. 6C, while the mount 310 is in an open operative state, locking rod 170 can be pushed distally (arrow 356 in FIGS. 5D and 6C), e.g., because locking rod 170 may be biased distally by spring 348. In some such implementations, and as shown, locking rod 170 is pushed distally such that it extends into anchor head 210, thereby locking the driver 104 to the anchor head. Consequently, in some implementations, motion of locking rod 170 into the anchor head can occur automatically when the driver 104 has been sufficiently advanced into the anchor head (e.g., such that the detent protrusions 312 move radially out of the lumen 153).

In some implementations, following anchoring of tissue anchor 202, when an operator wishes to pull the driver in a proximal direction, the operator can pull the release mechanism's finger-engager 344 (FIG. 5A) proximally, causing compression of spring 348 and retraction of locking rod 170 in a proximal direction, out of drive head 152. This may be carried out concurrently with retraction of driver 104, e.g., in a proximal direction. In some implementations, as driver 104 is pulled in a proximal direction, leading surface 318 of mount 310 can disengage proximal surface 134 of the tissue anchor, removing the deflection from portions of mount 310 and causing the mount to return to its initial position. Consequently, detent protrusions 312 return to their initial position within slots 316, and once again obstruct locking rod 170 from being pushed in a distal direction. Once the detent protrusions 312 are back in their initial position, the operator may release finger-engager 334, and the locking rod will remain out of the drive head.

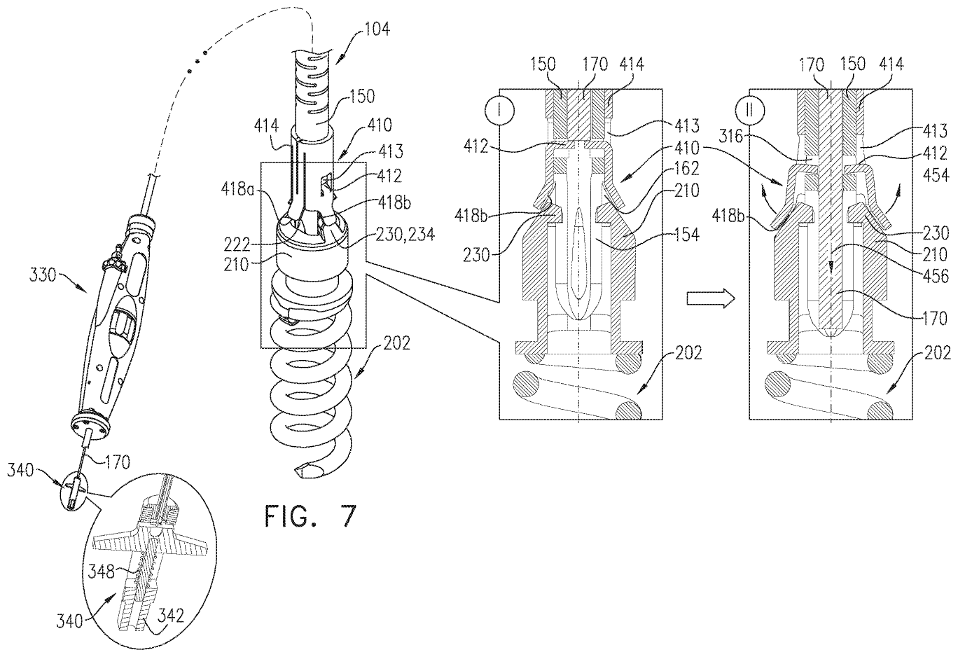

Reference is now made to FIG. 7, which is a perspective view illustration and a sectional illustration of an apparatus 400. Apparatus 400 is substantially similar to apparatus 300 described hereinabove with respect to FIGS. 5A to 6C, and includes handle 330, anchor release mechanism 340, driver 104, tissue anchor 202, and locking rod 170, substantially as described hereinabove. Apparatus 400 includes a mount 410, similar to mount 310 described hereinabove with respect to FIGS. 5A to 6C, and which operates in a similar manner.

In some implementations, mount 410 is formed of a unitary tube, which may be a laser cut hypotube, and differs from mount 310 of FIGS. 5A to 6C in a few structural details. Specifically, as shown in FIG. 7, detent protrusions 412 of mount 410 can be formed by cutting portions of cylindrical body 414 of the mount, and bending them inward in a medial direction. Consequently, mount 410 includes portals 413 in the cylindrical body 414.

In some implementations, cylindrical body 414 of mount 410 defines leading surfaces 418a, 418b at a distal end thereof. In some implementations, and similarly to as shown regarding leading surfaces 318 in FIG. 6A-C, leading surfaces 418a, 418b are beveled, and are formed by bending portions of the unitary tube in a radial direction. In some implementations, beveled leading surface 418a, 418b are equidistantly distributed about the circumference of mount 410. As a result, leading surfaces 418a, 418b can assist in centering drive head 152 and mount 410 relative to anchor head 210.

In some implementations, cylindrical body 414 of mount 410 has a plurality of longitudinal slots 420 formed therein, e.g., as described hereinabove with respect to FIGS. 5A-D. In some implementations, and as shown in FIG. 7, two leading surfaces 418a are longitudinally aligned with a portion of cylindrical body 414 having slots 420 formed therein, and two leading surfaces 418b are longitudinally aligned with portals 413.

As seen in frame I of FIG. 7, detent protrusions 412 are disposed within slots 316 of shaft 150, and extend medially into lumen 153. As such, detent protrusions 412 obstruct locking rod 170, which in some implementations can be biased in a distal direction toward drive head 152, from being pushed into the drive head. Consequently, this arrangement of mount 410 may be considered a blocking operative state of the mount. In some implementations while the mount 410 is in a blocking operative state, engagement fingers 154 of driver 104 are disposed within cavity 222 of anchor head 210, and protrusions 230 are disposed within slots 162, as described hereinabove. As seen, at least two of leading surfaces 418a, 418b of mount 410 engage proximal surfaces 234 of protrusions 230.

In some implementations, as driver 104 advances further in a distal direction, leading surfaces 418a, 418b of mount 410 can slide over a proximal surface of anchor head 210, causing at least a portion of mount 410 to deflect or pivot radially outwardly in the direction of arrows 454, as shown in frame II of FIG. 7. In some implementations, and as shown, at least two leading surfaces 418a, 418b can slide over proximal surface 234 of protrusions 230. Deflection of mount 410 causes detent protrusions 412 to pivot, in a manner that withdraws detent protrusions 412 from obstructing the lumen 153 and/or the locking rod 170, such that the mount is now in an open operative state.

In some implementations, while in an open operative state, locking rod 170 can be pushed in a distal direction. For instance, the locking rod 170 can be pushed by spring 348 of anchor release mechanism 340 in a distal direction, indicated by arrow 456, such that the locking rod extends into anchor head 210, thereby locking the driver 104 to the anchor head. In some such implementations, when the locking rod is biased in a distal direction, motion of locking rod 170 into the anchor head can occur automatically when the driver 104 has been sufficiently advanced into the anchor head (e.g., such that the detent protrusions 412 no longer obstruct the lumen 153).

In some implementations, detachment of driver 104 from tissue anchor 202 is carried out by use of anchor release mechanism 340, substantially as described hereinabove with respect to FIGS. 5A to 6C.

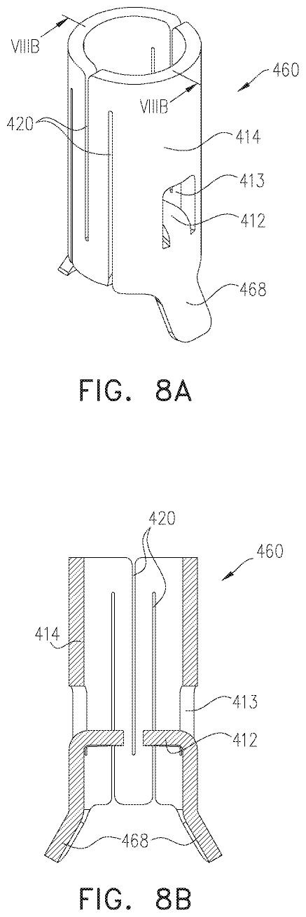

Reference is now made to FIGS. 8A and 8B, which are, respectively, a perspective view illustration and a sectional illustration of a mount 460 usable and/or suitable for use with the apparatus of FIGS. 5A to 7, In accordance with some implementations. Mount 460 is substantially similar to mount 410, and similarly includes detent protrusions 412, portals 413, cylindrical body 414, and longitudinal slots 420. Mount 460 differs from mount 410 in that it includes two leading surfaces 468, which are longitudinally aligned with portals 413, corresponding to leading surfaces 418b of FIG. 7.

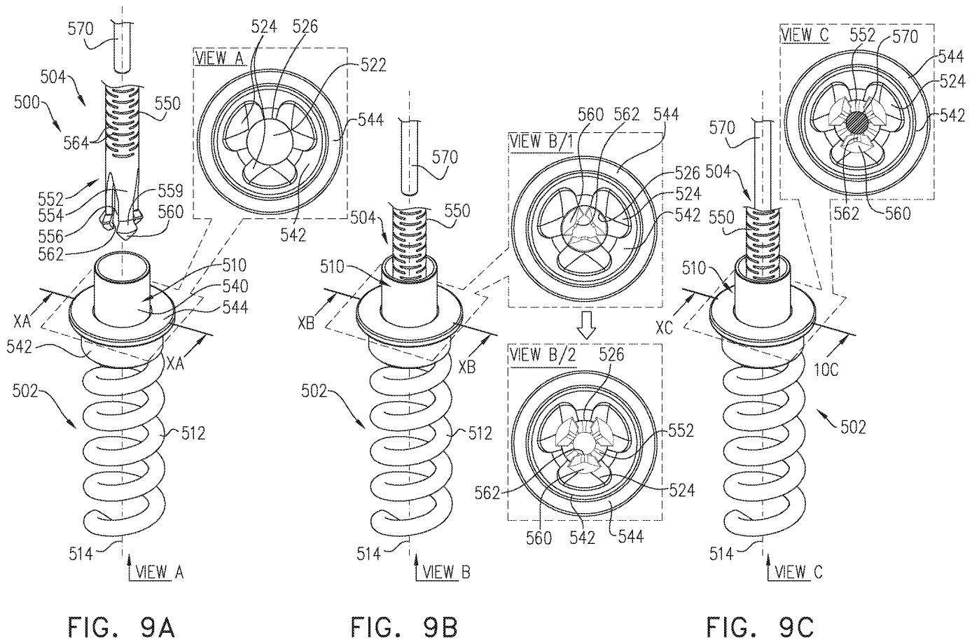

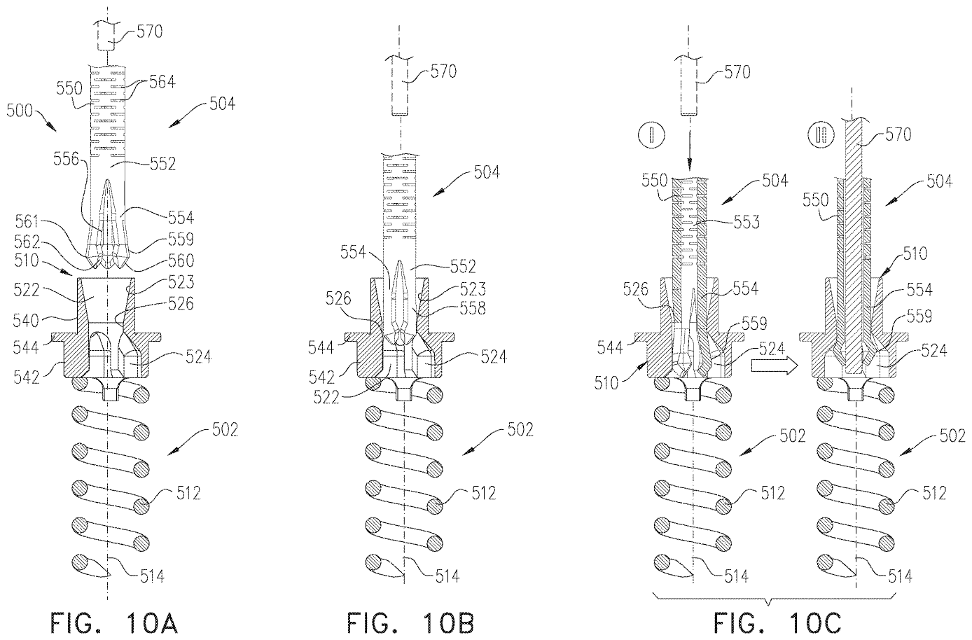

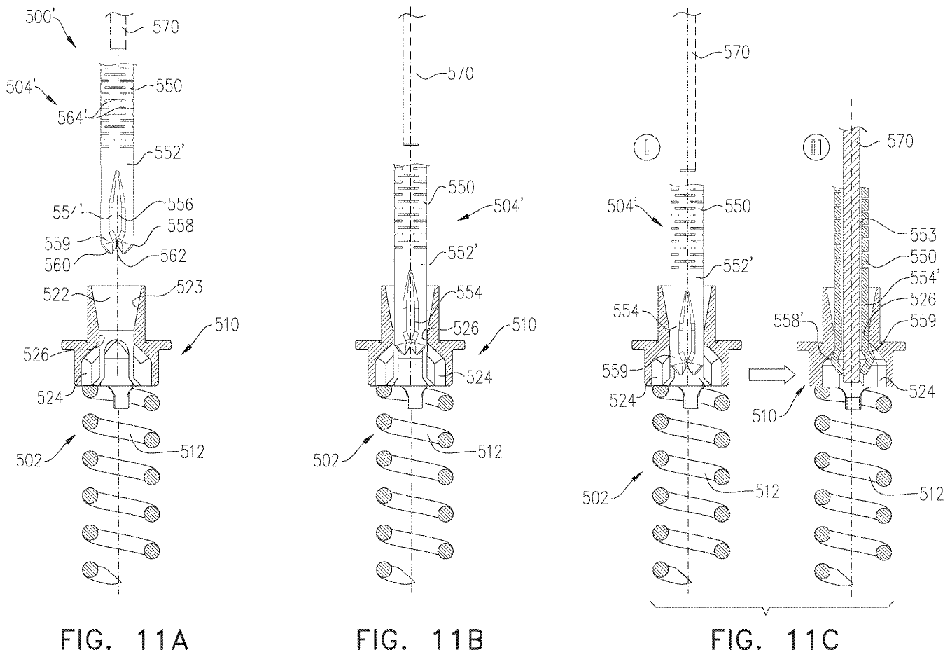

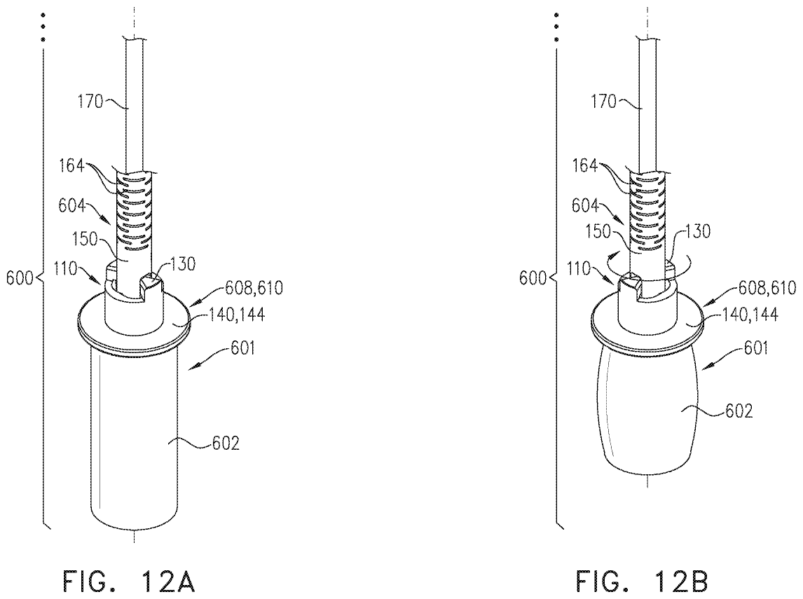

Reference is now made to FIGS. 9A, 9B, and 9C, which are perspective view illustrations of an apparatus 500 including a tissue anchor 502 and a driver 504, at different stages of connection therebetween, in accordance with some implementations. Reference is additionally made to FIGS. 10A, 10B, and 10C, which are sectional illustrations of apparatus 500 at corresponding stages of connection, in accordance with some implementations. The sectional illustrations of FIGS. 10A, 10B, and 10C, are taken along corresponding section lines XA-XA, XB-XB, and XC-XC in FIGS. 9A, 9B, and 9C, respectively.

As seen in FIGS. 9A and 10A, tissue anchor 502 includes an anchor head 510 and a helical tissue-engaging element 512, which extends away from anchor head 510 to define an anchor axis 514. Shaft 550 of driver 504 is configured to transluminally advance drive head 552, engaged with tissue anchor 502, to the heart of a subject (e.g., a living subject, a simulation, etc.), while a proximal part of the shaft remains outside of the subject's body.

Anchor head 510 includes an inner wall circumscribing a cavity 522, the cavity having a beveled surface 523 at a proximal end, and at least three lobes 524 at a distal end. A ridge 526 separates beveled surface 523 from lobes 524. Lobes 524 are seen clearly in view A of FIG. 9A, which is a planar bottom view of the apparatus.

In some implementations, an outer surface of anchor head 510 may include a proximal portion 540 having a first circumference, and a distal portion 542 having a second circumference. In some implementations, the second circumference can be greater than the first circumference, such that a shoulder is formed between proximal portion 540 and distal portion 542. In some implementations, and as illustrated, a flange 544 can extend radially out of the outer surface of anchor head 510, at the shoulder between proximal portion 540 and distal portion 542.

In some implementations, driver 504 includes a shaft 550 and a drive head 552 disposed at a distal end of the shaft. A lumen 553 extends longitudinally through shaft 550 and through drive head 552. Drive head 552 defines at least three longitudinal fingers 554, here illustrated as three fingers, separated by at least three longitudinal slots 556. Fingers 554 are adapted to engage respective lobes 524 within cavity 522 of anchor head 510.

In some implementations, a tip of each of fingers 554 comprises a tooth 558, which includes beveled surfaces 559 and 560 converging at a ridge 561, and a medially directed point 562. In some implementations, tooth 558 can have a curved, and/or have a triangular contour, as shown in FIG. 1A. In some implementations, teeth 558 may be formed by bending fingers 554 to have the illustrated structure. It is to be appreciated that torque is delivered from beveled surfaces 560 of teeth 558 of driver 504 to lobes 524 of tissue anchor 502, to facilitate the shaft driving rotation of the tissue anchor.

In some implementations, driver 504 comprises a unitary elongate tube that defines shaft 550 and drive head 552, such that a connection between the shaft and the drive head is devoid of welding and of adhesives. In some implementations, engagement fingers 554 are formed by laser cutting slots 556, separating the fingers, into the unitary elongate tube. In some implementations, the unitary elongate tube further includes a plurality of cuts 564, that configure shaft 550 as a flexible hypotube.

In some implementations, and as shown in FIG. 9A, and 10A, when no force is applied to drive head 552, a distal portion of drive head 552 has at least one external diameter which is greater than an external diameter of shaft 550. As shown in FIG. 9A, a diameter of drive head 552 at ridge 561 is greater than an external diameter of shaft 550.

FIGS. 9A and 10A illustrate apparatus 500 with driver 504 longitudinally aligned with and disposed proximally to tissue anchor 502, such that the driver and the tissue anchor are ready for engagement therebetween. Alternatively, apparatus 500 can be provided with driver 504 (e.g., drive head 552 thereof) already engaged to tissue anchor 502.

FIGS. 9B and 10B illustrate driver 504 having being advanced distally to engage anchor head 510. In the state shown in FIGS. 9B and 10B, a distal end of drive head 552 is disposed within cavity 522, such that teeth 558 of fingers 554 are deflected medially against proximal beveled surface 523 within cavity 522. View B/1 of FIG. 9B, which is a planar bottom view of the apparatus in the state corresponding to that of FIG. 10B, shows teeth 558 of drive head 552 disposed within the narrowest portion of cavity 522, at ridge 526. View B/2 of FIG. 9B, which is a planar bottom view of the apparatus in the following completion of advancement of drive head 552 distally into cavity 522, shows teeth 558 of drive head 552 being disposed within, and having engaged, respective lobes 524 of the cavity. This state is also illustrated in view I of FIG. 10C. In some implementations, teeth 558 snap radially into lobes 524, once drive head 552 advances distally beyond ridge 526.

FIG. 9C and view II of FIG. 10C illustrate apparatus 500 where driver 504 is engaged with tissue anchor 502. In the engaged state, teeth 558 of fingers 554 have snapped radially to engage lobes 524. Specifically, beveled surfaces 560 of the teeth engage corresponding beveled surfaces 559 of lobes 524.

In some implementations, and as shown, a longitudinal locking rod 570 is received within lumen 553 extending through driver 504. Presence of locking rod 570 within lumen 553 obstructs disengagement of drive head 552 from anchor head 510, by preventing medial deflection of teeth 558 to move past ridge 526.

In some implementations, for removal of driver 504 from tissue anchor 502, locking rod 570 is first removed from lumen 553. Subsequently, as shaft 550 is pulled in a proximal direction, fingers 554 deflect medially against beveled surfaces of lobes 524 until teeth 558 of the fingers disengage from the lobes, and pass ridge 526.

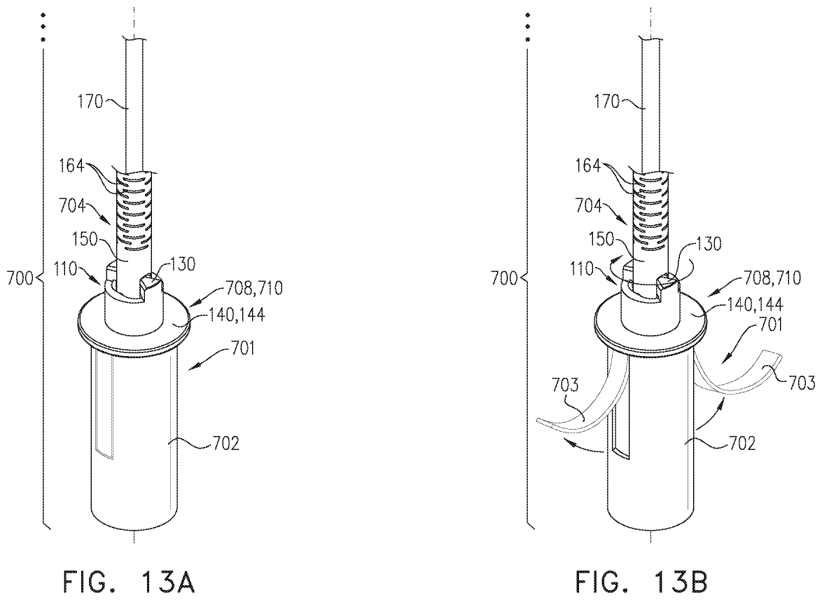

Reference is made to FIGS. 11A, 11B, and 11C, which are sectional illustrations of an apparatus 500′ in stages of connection corresponding to FIG. 10A-C, in accordance with some implementations. Apparatus 500′ is similar to apparatus 500, including tissue anchor 502, a driver 504′, and locking rod 570, substantially as described hereinabove with respect to FIGS. 9A to 10C.

Similarly to apparatus 500, shaft 550 of driver 504′ is configured to transluminally advance drive head 552′, engaged with tissue anchor 502, to the heart of a subject, while a proximal part of the shaft remains outside of the subject's body.

Apparatus 500′ of FIGS. 11A to 11C differs from apparatus 500 in that, when no force is applied to drive head 552′, fingers 554′ of apparatus 500′ do not deflect radially outwardly relative to shaft 550 thereof. As such, when no force is applied to drive head 552′, an external diameter of the drive head is equal to, or smaller than, an external diameter of shaft 550.

FIG. 11A illustrates apparatus 500′ with driver 504′ longitudinally aligned with and disposed proximally to tissue anchor 502, and ready for engagement therebetween. Alternatively, apparatus 500′ can be provided with driver 504 (e.g., drive head 552′ thereof) already engaged to tissue anchor 502.

FIG. 11B illustrates driver 504′ having been advanced distally to engage anchor head 510. As shown, a distal end of drive head 552′ is disposed within cavity 522, at ridge 526 thereof, without requiring any deflection of fingers 554′. View B/1 of FIG. 9B, which is a planar bottom view of the apparatus in the state corresponding to that of FIG. 10B, corresponds to the state of apparatus 500′ as shown in FIG. 11B.

View I of FIG. 11C shows driver 504′ fully advanced into cavity 522, while fingers 554′ continue to remain centered, and do not engage lobes 524. This state continues to correspond to that shown in view B/1 of FIG. 9B.

In view II of FIG. 11C, longitudinal locking rod 570 is received within lumen 553 extending through driver 504′. As shown in this example, presence of locking rod 570 within lumen 553 pushes fingers 554′ radially outwardly, causing radial deflection of teeth 558 toward lobes 524 of anchor head 510. In this way, beveled surfaces 560 of the teeth engage corresponding beveled surfaces 559 of lobes 524. It is to be appreciated that rotational torque is delivered from beveled surfaces 560 of teeth 558 of driver 504′ to lobes 524 of tissue anchor 502, to facilitate the shaft driving rotation of the tissue anchor.

In some implementations, for removal of driver 504′ from tissue anchor 502, locking rod 570 is first removed from lumen 553. Consequently, fingers 554′ deflect medially back to their position as shown in illustration I of FIG. 11C, disengaging from the surfaces of lobes 524 and enabling shaft 550 to be pulled in a proximal direction, and enabling fingers 554′ to pass ridge 526.

Reference is made to FIG. 12A-B and 13A-B, which are schematic illustrations showing apparatus 600, 700, in accordance with some implementations. As shown, apparatus 600, 700 comprises an implant driver 604 that is substantially identical to driver 104, as well as an implant 601, 701 comprising an interface 610, 710 that is substantially identical to anchor head 110 of tissue anchor 102.

Implants 601, 701 are schematic examples of implants to which implant driver 604, 704 can be used to transfer torque. In some implementations, and as shown, implant 601, 701 defines an implant body 602, 702 that extends away from interface 610, 710, e.g., which is disposed at the implant's head. For example, and as shown, the implant's interface 610, 710 can be identical to anchor head 110 described hereinabove, mutatis mutandis.

In some implementations, and similarly to driver 104, implant driver 604, 704 can be used to transfer torque to the implant's interface 610, 710, e.g., thereby actuating implant 601, 701. Alternatively, the implant driver and the interface of apparatus 600, 700 can comprise any of the pairs of drivers 104, 504 and anchor heads 210, 510 (e.g., including funnel 270 and/or mount 460) described hereinabove, mutatis mutandis.

In some implementations, implant driver 604, 704 is used to advance (e.g., advance transluminally) implant 601, 701 to an anatomical site via a delivery tool (not shown). In some such implementations, apparatus 600, 700 can be provided with implant driver 604, 704 (e.g., drive head 152 thereof) already engaged with interface 610, 710. In this way, implant 601, 701 can be advanced to the anatomical site while implant driver 604, 704 is engaged with interface 610, 710.

In some implementations, implant 601, 701 comprises an implant-modification mechanism (IMM) 608, 708 that is actuated by receiving torque from implant driver 604, 704 at interface 610, 710. In some such implementations, actuating IMM 608, 708 changes a conformation of implant 601, 701, e.g., such that the implant assumes an actuation-conformation. For example, implant 601, 701 can retain the actuation-conformation after receiving the torque from implant driver 604, 704, e.g., after the implant driver disengages interface 610, 710. In some such implementations, actuating IMM 608, 708 causes implant 601, 701 to extrude a substance (e.g., a therapeutic substance) to the anatomical site (e.g., into a body cavity) at which the implant is disposed.

In some implementations, actuating IMM 608, 708 can change the implant's conformation (e.g., a conformation of implant body 602, 702) by changing a shape and/or dimension of the implant. In some such implementations, and as shown in the transition between FIG. 12A-B, applying torque to IMM 608 can change the length and/or width of implant body 602 by transferring torque to the implant. Alternatively or in addition, and as shown in the transition between FIG. 13A-B, implant 701 can define appendages 703 (e.g., limbs, barbs, spikes, etc.) that move when IMM 708 transfers torque to the implant. For example, and as shown, appendages 703 can be initially disposed within implant body 702, and applying torque to IMM 708 can expose the appendages from the implant body.

Each of the following publications is incorporated herein by reference. Any of the drivers described in any of these publications may be replaced with any of the drivers described in the present disclosure, or may be modified to include any component or feature of any of the drivers described in the present disclosure, mutatis mutandis. Any of the drivers described in the present disclosure may be used to perform any of the techniques described in these publications, mutatis mutandis:

US Patent Application Publication 2012/0022557 to Cabiri et al.
US Patent Application Publication 2014/0309661 to Sheps et al.
US Patent Application Publication 2015/0272734 to Sheps et al.
US Patent Application Publication 2018/0049875 to Iflah et al.
US Patent Application Publication 2021/0145584 to Kasher et al.
International Patent Application Publication WO 2022/064401 to Halabi et al.
International Patent Application Publication WO 2022/172149 to Shafigh et al.

Reference is again made to FIGS. 1A-11C. For the purposes of this specification and the claims that follow, the term “substantially” is defined as “at least 95%” of the related quantity. For example, “substantially perpendicular” means at least 95% perpendicular, or having an angle in the range of 85° to 95°.

It should be understood that the use of “and/or” is defined inclusively such that the term “a and/or b” should be read to include the sets: “a and b,” “a or b,” “a,” “b.”

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

The techniques, methods, operations, steps, etc. described or suggested herein or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

The present disclosure is not limited to what has been particularly shown and described hereinabove. Rather, the scope of the present disclosure includes both combinations and subcombinations of the various features described hereinabove, as well as variations and modifications thereof that are not in the prior art, which would occur to persons skilled in the art upon reading the foregoing description. Further, the techniques, methods, operations, steps, etc. described or suggested herein can be performed on a living animal or on a non-living simulation, such as on a cadaver, cadaver heart, simulator (e.g., with the body parts, tissue, etc. being simulated), etc.

Although the operations of some of the disclosed examples are described in a particular, sequential order for convenient presentation, it should be understood that this manner of description encompasses rearrangement, unless a particular ordering is required by specific language set forth above. For example, operations or steps described sequentially can in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms can vary depending on the particular implementation and are discernible by one of ordinary skill in the art.

Example Implementations (some non-limiting examples of the concepts herein are recited below):
Example 1. Apparatus for use with a heart of a subject, the apparatus comprising: a tissue anchor, having an anchor head and a tissue-engaging element that extends away from the anchor head to define an anchor axis of the tissue anchor, the anchor head having: an engagement surface, and/or proximally from the engagement surface, one or more protrusions that extend medially; and/or a driver comprising a shaft and a drive head, the drive head: disposed at a distal end of the shaft, defining one or more engagement fingers, each of the engagement fingers having a root, and a tip distal from the root, and/or configured to engage the anchor head such that, for each of the engagement fingers, the engagement finger extends distally beyond the one or more protrusions of the anchor head, such that: the tip is engaged with the engagement surface in a manner that inhibits axial movement of the drive head relative to the anchor head, and/or the root is disposed against a respective protrusion of the anchor head in a manner that delivers torque to the anchor head upon rotation of the shaft.
Example 2. The apparatus according to example 1, wherein at least one of the tissue anchor and the driver is sterile.
Example 3. The apparatus according to example 1, wherein the driver defines two engagement fingers, the engagement fingers being parallel to each other and opposite each other.
Example 4. The apparatus according to any one of examples 1-3, wherein the anchor head includes a wall circumscribing a cavity, the wall having a distal beveled surface forming the engagement surface.
Example 5. The apparatus according to example 4, wherein the wall has a proximal beveled surface proximal to the distal beveled surface.
Example 6. The apparatus according to example 5, wherein, during insertion of the drive head into the anchor head, the tip of each of the engagement fingers deflects medially by the proximal beveled surface, and snaps radially to engage the distal beveled surface.
Example 7. The apparatus according to example 6, wherein, for removal of the driver from the tissue anchor, the tip of each of the engagement fingers deflects medially against the distal beveled surface until the tip disengages from the engagement surface.
Example 8. The apparatus according to any one of examples 1-7, wherein the one or more protrusions are disposed at a proximal end of the anchor head.
Example 9. The apparatus according to any one of examples 1-8, wherein the driver defines a lumen extending longitudinally through the shaft and through the drive head.
Example 10. The apparatus according to any one of examples 1-9, wherein the one or more protrusions of the anchor head are substantially triangular and have a narrower portion disposed medially to a wider portion thereof.
Example 11. The apparatus according to any one of examples 1-10, wherein a proximal surface of each of the one or more protrusions is substantially planar.
Example 12. The apparatus according to any one of examples 1-10, wherein a proximal surface of each of the one or more protrusions comprises a beveled portion, the beveled portion configured to assist in aligning the driver relative to the anchor head.
Example 13. The apparatus according to any one of examples 1-12, wherein the driver comprises a unitary elongate tube that defines the drive head and the shaft.
Example 14. The apparatus according to example 13, wherein the one or more fingers of the driver are formed by laser cutting at least one slot into the unitary elongate tube.
Example 15. The apparatus according to any one of examples 13-14, wherein the tube includes a plurality of cuts that configure the shaft as a flexible hypotube.
Example 16. The apparatus according to any one of examples 13-15, wherein each of the engagement fingers comprises a cut-out, proximal to the tip thereof, the cut-out being laser cut into the unitary elongate tube.
Example 17. The apparatus according to any one of examples 1-16, wherein a connection between the shaft and the drive head of the driver is devoid of welding and of adhesives.
Example 18. The apparatus according to any one of examples 1-17, wherein, when no force is applied to the drive head, an external diameter of the drive head at the tips of the engagement fingers is not greater than an external diameter of the shaft.
Example 19. The apparatus according to any one of examples 1-18, further comprising a locking rod received in a lumen extending through the driver, such that a presence of the locking rod within the drive head obstructs disengagement of the drive head from the anchor head.
Example 20. The apparatus according to example 19, wherein the driver further includes: a spring, adapted to push the locking rod toward being disposed within the drive head; a detent, at a distal part of the driver, adapted to obstruct the locking rod from being pushed into the drive head; and/or a mount on which the detent is mounted, the mount: coupled to the shaft, having a leading surface, and/or adapted such that advancing the drive head into engagement with the anchor head slides the leading surface over the anchor head in a manner that withdraws the detent from obstructing the locking rod.
Example 21. The apparatus according to example 20, wherein a surface of the anchor head, distal to the tissue-engaging element, has beveled wall section, and wherein the leading surface of the mount is adapted to slide over the beveled wall section.
Example 22. The apparatus according to any one of examples 20-21, wherein the leading surface of the mount is a beveled surface.
Example 23. The apparatus according to any one of examples 20-22, wherein the mount has a blocking operative state, in which the detent obstructs the locking rod from being pushed into the drive head, and an open operative state, in which the detent is withdrawn from obstructing the locking rod.
Example 24. The apparatus according to any one of examples 20-23, wherein sliding of the leading surface of the mount over the anchor head causes portions of the mount, onto which the detent is mounted, to pivot radially outwardly, to withdraw the detent from obstructing the locking rod.
Example 25. The apparatus according to any one of examples 20-24, wherein the detent comprises at least one protrusion, received in a slot at the distal part of the driver, such that the protrusions are disposed within the lumen and obstruct the locking rod.
Example 26. The apparatus according to any one of examples 1-25, further comprising a funnel, having a cylindrical exterior surface and a beveled interior surface, the funnel being attachable to a proximal portion of the anchor head and being configured to receive the driver and to guide the driver to the anchor head, for engagement therebetween.
Example 27. An apparatus, comprising: a tissue anchor including a tissue-engaging element and an anchor head; a driver, comprising: a shaft defining a lumen, a drive head fixed to a distal end of the shaft, and configured to reversibly engage the anchor head; a locking rod, extending from a proximal part of the driver through the lumen, and adapted such that a presence of the locking rod within the drive head obstructs disengagement of the drive head from the anchor head; a spring, adapted to push the locking rod toward being disposed within the drive head; a detent, at a distal part of the driver, adapted to obstruct the locking rod from being pushed into the drive head; and/or a mount on which the detent is mounted, the mount: coupled to the shaft, having a leading surface, and/or adapted such that advancing the drive head into engagement with the anchor head slides the leading surface over the anchor head in a manner that withdraws the detent from obstructing the locking rod.
Example 28. The apparatus according to example 27, wherein at least one of the tissue anchor and the driver is sterile.
Example 29. The apparatus according to example 27, wherein a surface of the anchor head, distal to the tissue-engaging element, has beveled wall section, and wherein the leading surface of the mount is adapted to slide over the beveled wall section.
Example 30. The apparatus according to any one of examples 27-29, wherein the leading surface of the mount is a beveled surface.
Example 31. The apparatus according to any one of examples 27-30, wherein the mount has a blocking operative state, in which the detent obstructs the locking rod from being pushed into the drive head, and an open operative state, in which the detent is withdrawn from obstructing the locking rod.
Example 32. The apparatus according to any one of examples 27-31, wherein sliding of the leading surface of the mount over the anchor head causes portions of the mount, onto which the detent is mounted, to pivot radially outwardly, to withdraw the detent from obstructing the locking rod.
Example 33. The apparatus according to any one of examples 27-32, wherein the detent comprises one or more protrusion, received in a slot at the distal part of the driver, such that each protrusion is disposed within the lumen and obstructs the locking rod.
Example 34. The apparatus according to example 33, wherein the tissue-engaging element extends away from the anchor head to define an anchor axis of the tissue anchor, and wherein the anchor head has an engagement surface, and, proximally from the engagement surface, the one or more protrusions extend medially.
Example 35. The apparatus according to example 34, wherein the drive head of the driver is disposed at a distal end of the shaft, defines one or more engagement fingers, each of the engagement fingers having a root, and a tip distal from the root, and is configured to engage the anchor head, wherein, for each of the engagement fingers, the engagement finger extends distally beyond the one or more protrusions of the anchor head, such that: the tip is engaged with the engagement surface in a manner that inhibits axial movement of the drive head relative to the anchor head, and/or the root is disposed against a respective protrusion of the anchor head in a manner that delivers torque to the anchor head upon rotation of the shaft.
Example 36. The apparatus according to example 35, wherein the driver defines two engagement fingers, the engagement fingers being parallel to each other and opposite each other.
Example 37. The apparatus according to any one of examples 35-36, wherein the anchor head includes a wall circumscribing a cavity, the wall having a distal beveled surface forming the engagement surface.
Example 38. The apparatus according to example 37, wherein the wall has a proximal beveled surface proximal to the distal beveled surface.
Example 39. The apparatus according to example 38, wherein, during insertion of the drive head into the anchor head, the tip of each of the engagement fingers deflects medially by the proximal beveled surface, and snaps radially to engage the distal beveled surface.
Example 40. The apparatus according to example 39, wherein, for removal of the driver from the tissue anchor, the tip of each of the engagement fingers deflects medially against the distal beveled surface until each of the tips disengage from the engagement surface.
Example 41. The apparatus according to any one of examples 36-40, wherein the one or more engagement fingers of the driver are formed by laser cutting at least one slot into a unitary elongate tube.
Example 42. The apparatus according to example 41, wherein each of the engagement fingers comprises a cut-out, proximal to the tip thereof, the cut-out being laser cut into the unitary elongate tube.
Example 43. The apparatus according to any one of examples 36-42, wherein, when no force is applied to the drive head, an external diameter of the drive head at the tips of each of the engagement fingers is not greater than an external diameter of the shaft.
Example 44. The apparatus according to any one of examples 35-43, wherein the one or more protrusions are disposed at a proximal end of the anchor head.
Example 45. The apparatus according to any one of examples 35-44, wherein the one or more protrusions are substantially triangular and have a narrower portion disposed medially to a wider portion thereof.
Example 46. The apparatus according to any one of examples 35-45, wherein a proximal surface of each of the protrusions is substantially planar.
Example 47. The apparatus according to any one of examples 35-45, wherein a proximal surface of each of the protrusions has at least a beveled portion, the beveled portion configured to assist in aligning the driver relative to the anchor head.
Example 48. The apparatus according to any one of examples 27-47, wherein the shaft includes a plurality of cuts that configure the shaft as a flexible hypotube.
Example 49. The apparatus according to any one of examples 27-48, wherein the drive head and the shaft are formed of a unitary elongate tube, and wherein the shaft is configured to transluminally advance the drive head, engaged with the tissue anchor, to a heart of a subject while a proximal part of the shaft remains outside of the subject.
Example 50. The apparatus according to any one of examples 27-49, wherein a connection between the shaft and the drive head of the driver is devoid of welding and of adhesives.
Example 51. The apparatus according to any one of examples 27-50, further comprising a funnel, having a cylindrical exterior surface and a beveled interior surface, the funnel being attachable to a proximal portion of the anchor head and being configured to receive the driver and to guide the driver to the center of the anchor head, for engagement therebetween.
Example 52. Apparatus for use with a heart of a subject, the apparatus comprising: a driver adapted to engage an anchor head of a tissue anchor, the driver comprising a unitary elongate tube that defines: a drive head, defining at least three longitudinal fingers separated by at least three longitudinal slots, and configured to engage the anchor head by each of the longitudinal fingers engaging with a respective portion of the anchor head; and/or a shaft, configured to transluminally advance the drive head, engaged with the tissue anchor, to the heart while a proximal part of the shaft remains outside of the subject.
Example 53. The apparatus according to example 52, further comprising the tissue anchor.
Example 54. The apparatus according to any one of examples 52-53, wherein at least one of the tissue anchor and the driver is sterile.
Example 55. The apparatus according to any one of examples 52-53, further comprising a locking rod received in a lumen of the shaft to prevent detachment of the driver from the anchor head.
Example 56. The apparatus according to any one of examples 53-55, wherein the anchor head defines a cavity having at least three lobes, and wherein each of the at least three longitudinal fingers of the driver engages with one of the at three lobes.
Example 57. The apparatus according to any one of examples 52-56, wherein the at least three longitudinal slots of the driver are laser cut into the unitary elongate tube to form the drive head.
Example 58. The apparatus according to any one of examples 52-57, wherein a distal end of each of the at least three longitudinal fingers of the driver comprises a radially inward facing tooth.
Example 59. The apparatus according to any one of examples 52-58, wherein the shaft and the drive head of the driver are connected, the connection between the shaft and the drive head being devoid of welding and of adhesives.
Example 60. The apparatus according to any one of examples 52-59, wherein the tube includes a plurality of cuts that configure the shaft as a flexible hypotube.
Example 61. The apparatus according to any one of examples 55-60, wherein, when no force is applied to the drive head of the driver, an external diameter of the drive head is equal to, or smaller than, an external diameter of the shaft.
Example 62. The apparatus according to example 61, wherein upon insertion of the drive head into the anchor head of the tissue anchor the drive head remains disengaged from the anchor head, and upon insertion of the locking rod into the drive head the longitudinal fingers of the drive head are pushed radially outwardly to engage the anchor head.
Example 63. The apparatus according to example 62, wherein removal of the locking rod from the lumen of the driver causes the longitudinal fingers to disengage from the anchor head.
Example 64. The apparatus according to any one of examples 55-60, wherein, when no force is applied to the drive head of the driver, an external diameter of at least a distal end the drive head is greater than an external diameter of the shaft.
Example 65. The apparatus according to example 64, wherein, when the driver is disposed within the anchor head, the longitudinal fingers of the driver snap fit into a cavity in the anchor head for engagement therebetween, and wherein the locking rod prevents medial motion of the longitudinal fingers, thereby preventing disengagement of the longitudinal fingers from the anchor head.
Example 66. The apparatus according to example 65, wherein, following removal of the locking rod from the lumen of the driver, the longitudinal fingers can disengage from the anchor head by deflecting medially against beveled surface of the anchor head.
Example 67. A method of manufacture of a driver, the method comprising: cutting at least three longitudinal slots in one end of an elongate tube, to define at least three longitudinal fingers; and/or forming a plurality of cuts in the remainder of the elongate tube, to configure the remainder of the elongate tube as a flexible hypotube, wherein the end of the elongate tube including the at least three longitudinal slots comprises a drive head or the driver, and the remainder of the elongate tube comprises a shaft of the driver.
Example 68. The method according to example 67, further comprising bending a tip of each of the at least three longitudinal fingers to form a point that extends medially.
Example 69. The method according to any one of examples 67-68, wherein cutting of the at least three longitudinal slots and/or forming of the plurality of cuts comprises laser cutting the elongate tube.
Example 70. The method according to any one of examples 67-69, further comprising deflecting an end of each of the at least three fingers in a radial direction, such that a circumference of the driver at the drive head is greater than a circumference of the shaft.
Example 71. The method according to any one of examples 67-70, wherein the method is devoid of welding and of adhering.
Example 72. The method according to any one of examples 67-71, further comprising sterilizing the driver.
Example 73. Apparatus for use with a heart of a subject, the apparatus comprising: a driver comprising: a shaft; and/or a drive head disposed at a distal end of the shaft, the drive head comprising: at least two fingers and at least one slot disposed between the at least two fingers, wherein the driver is configured to mate with an anchor head of a tissue anchor such that: during insertion of the driver into the anchor head, the at least two fingers are deflected laterally in a manner that inhibits axial movement of the drive head relative to the anchor, and/or the at least one slot abuts a protrusion of the anchor head in a manner that delivers torque to the anchor head upon rotation of the shaft.
Example 74. The apparatus according to example 73, wherein the driver is sterile.
Example 75. Apparatus for use with a tissue of a subject, the apparatus comprising: an implant having an implant head and an implant body that extends away from the implant head, the implant head having: an engagement surface, and/or proximally from the engagement surface, one or more protrusions that extend medially; and/or an implant driver comprising a shaft and a drive head, the drive head: disposed at a distal end of the shaft, defining one or more engagement fingers, each of the engagement fingers having a root, and a tip distal from the root, and/or configured to engage the implant head such that, for each of the engagement fingers, the engagement finger extends distally beyond the one or more protrusions of the implant head, such that: the tip is engaged with the engagement surface in a manner that inhibits axial movement of the drive head relative to the implant head, and/or the root is disposed against a respective protrusion of the implant head in a manner that transfers torque to the implant head upon rotation of the shaft.
Example 76. The apparatus according to example 75, wherein at least one of the implant and the implant driver is sterile.
Example 77. The apparatus according to any one of examples 75-77, wherein the implant is configured such that transfer of torque to the implant head actuates the implant.
Example 78. The apparatus according to example 77, wherein the implant is configured such that transfer of torque to the implant head modifies a conformation of the implant into an actuation conformation.
Example 79. The apparatus according to example 78, wherein the implant is configured to retain the actuation conformation following transfer of torque to the implant head.
Example 80. The apparatus according to example 79, wherein: the implant driver is configured to, following transfer of the torque to the implant head, disengage the implant head, and/or the implant is configured to retain the actuation conformation following disengagement of the implant driver from the implant head.
Example 81. Apparatus for use with a subject, the apparatus comprising: an implant, configured to be delivered into the subject, and comprising: an implant-modification mechanism (IMM): configured to, upon actuation thereof, modify a conformation of the implant, and/or defining an interface: operatively coupled to the IMM such that torque applied to the interface actuates the IMM, having an engagement surface, and/or having, proximally from the engagement surface, one or more protrusions that extend medially; and/or a delivery tool, configured to deliver the implant into the subject, and comprising a driver that comprises a shaft and a drive head, the drive head: disposed at a distal end of the shaft, defining one or more engagement fingers, each of the engagement fingers having a root, and a tip distal from the root, and/or configured to engage the interface such that, for each of the engagement fingers, the engagement finger extends distally beyond the one or more protrusions of the interface, such that: the tip is engaged with the engagement surface in a manner that inhibits axial movement of the drive head relative to the interface, and/or the root is disposed against a respective protrusion of the interface in a manner that delivers the torque to the interface upon rotation of the shaft.
Example 82. The apparatus according to example 81, wherein at least one of the implant and the delivery tool is sterile.

The present invention is not limited to the examples that have been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

What is claimed is:

1. An apparatus for use with a heart of a subject, the apparatus comprising:
a driver comprising:
a shaft; and
a drive head disposed at a distal end of the shaft, the drive head comprising: at least two fingers and at least one slot disposed between the at least two fingers, wherein the driver is configured to engage an anchor head of a tissue anchor such that:
during insertion of the driver into the anchor head, the at least two fingers are deflected radially in a manner that inhibits axial movement of the drive head relative to the anchor, and
the at least one slot abuts a protrusion of the anchor head in a manner that delivers torque to the anchor head upon rotation of the shaft.

2. The apparatus according to claim 1, wherein the driver defines two fingers, the fingers being parallel to each other and opposite each other.

3. The apparatus according to claim 1, wherein a connection between the shaft and the drive head of the driver is devoid of welding and of adhesives.

4. The apparatus according to claim 1, wherein the apparatus further comprises the tissue anchor, the tissue anchor having an anchor head and a tissue-engaging element that extends away from the anchor head to define an anchor axis of the tissue anchor.

5. The apparatus according to claim 4, wherein the anchor head defines:
an engagement surface, and
proximally from the engagement surface, one or more protrusions that extend medially.

6. The apparatus according to claim 5, wherein the one or more protrusions are disposed at a proximal end of the anchor head.

7. The apparatus according to claim 5, wherein the one or more protrusions of the anchor head are substantially triangular and have a narrower portion disposed medially to a wider portion thereof.

8. The apparatus according to claim 5, wherein a proximal surface of each of the one or more protrusions is substantially planar.

9. The apparatus according to claim 5, wherein a proximal surface of each of the one or more protrusions comprises a beveled portion, the beveled portion configured to assist in aligning the driver relative to the anchor head.

10. The apparatus according to claim 5, wherein:
the drive head defines a root at a proximal portion of the slot, and each of the fingers defines a respective tip, distal from the root; and
the driver is configured to engage the anchor head such that, each of the fingers extends distally beyond the one or more protrusions of the anchor head, such that:
during insertion of the driver into the anchor head, the respective tips are deflected radially such that each tip is engaged with the engagement surface in a manner that inhibits axial movement of the drive head relative to the anchor head, and
the root is disposed against a respective protrusion of the anchor head in a manner that delivers torque to the anchor head upon rotation of the shaft.

11. The apparatus according to claim 10, wherein, when no force is applied to the drive head, an external diameter of the drive head at the tips of the fingers is not greater than an external diameter of the shaft.

12. The apparatus according to claim 10, wherein the anchor head includes a wall circumscribing a cavity, the wall having a distal beveled surface forming the engagement surface.

13. The apparatus according to claim 12, wherein the wall has a proximal beveled surface proximal to the distal beveled surface.

14. The apparatus according to claim 13, wherein, during insertion of the drive head into the anchor head, the tip of each of the fingers deflects medially by the proximal beveled surface, and snaps radially to engage the distal beveled surface.

15. The apparatus according to claim 14, wherein, for removal of the driver from the tissue anchor, the tip of each of the fingers deflects medially against the distal beveled surface until the tip disengages from the engagement surface.

16. The apparatus according to claim 1, wherein the driver comprises a unitary elongate tube that defines the drive head and the shaft.

17. The apparatus according to claim 16, wherein the one or more fingers of the driver are formed by laser cutting at least one slot into the unitary elongate tube.

18. The apparatus according to claim 16, wherein each of the fingers comprises a cut-out, proximal to a distal tip thereof, the cut-out being laser cut into the unitary elongate tube.

19. The apparatus according to claim 1, further comprising a locking rod received in a lumen extending through the driver, such that a presence of the locking rod within the drive head obstructs disengagement of the drive head from the anchor head.

20. The apparatus according to claim 19, wherein the driver further includes:
a spring, adapted to push the locking rod toward being disposed within the drive head;
a detent, at a distal part of the driver, adapted to obstruct the locking rod from being pushed into the drive head; and
a mount on which the detent is mounted, the mount:
coupled to the shaft,
having a leading surface, and
adapted such that advancing the drive head into engagement with the anchor head slides the leading surface over the anchor head in a manner that withdraws the detent from obstructing the locking rod.
